(19) United States
(12) Patent Application Publication
(10) Pub. No.: US 2026/0232589 A1
(43) Pub. Date: Aug. 13, 2026

(54) NANOPREPARATIONS LOADED WITH TRABECTEDIN AND PACLITAXEL

(71) Applicant: SHANGHAI JIAO TONG UNIVERSITY, Shanghai (CN)

(72) Inventors: Zixiu DU, Shanghai (CN); Wei WEI, Shanghai (CN); Wei BAO, Shanghai (CN); Xuexin ZHOU, Shanghai (CN)

(21) Appl. No.: 19/362,653

(22) Filed: Oct. 20, 2025

Related U.S. Application Data
(63) Continuation of application No. PCT/CN2024/076210, filed on Feb. 6, 2024.

(30) Foreign Application Priority Data
Feb. 21, 2023 (CN) 202310146703.4
Mar. 20, 2023 (CN) 202310273690.7

(51) Int. Cl. A61K 9/1273; A61K 31/337; A61K 31/472; A61K 47/34

(52) U.S. Cl. CPC A61K 9/1273; A61K 31/337; A61K 31/472; A61K 47/34

(57) ABSTRACT

A nanopreparation loaded with trabectedin and paclitaxel is provided. The nanopreparation loaded with trabectedin and paclitaxel includes a non-targeted drug-loaded micelle loaded with paclitaxel and a first targeting group linked to a first micelle carrier, where the non-targeted drug-loaded micelle has a first shell-core structure, and the first micelle carrier is prepared from poly(ε-caprolactone)-poly(2-ethyl-2-oxazoline) (PCL-PEOz), where a hydrophobic block of PCL and paclitaxel jointly form a core of the first shell-core structure, and a hydrophilic block of PEOz forms a shell of the first shell-core structure; and the first targeting group is linked via an amide bond formed between —NH₂ on a surface of the first targeting group and —COOH at a terminus of the PEOz on a surface of the shell.

### CROSS REFERENCE TO RELATED APPLICATION

This patent application is a Continuation of International Patent Application No. PCT/CN2024/076210, filed on Feb. 6, 2024, which claims priority to Chinese Patent Application Nos. 202310146703.4 (filed on Feb. 21, 2023) and 202310273690.7 (filed on Mar. 20, 2023), both filed with the China National Intellectual Property Administration. The disclosures of the three applications each are incorporated herein by reference in its entirety.

### TECHNICAL FIELD

The present disclosure belongs to the technical field of drug delivery, and relates to a hydrophobic drug nanopreparation, in particular to nanopreparations loaded with trabectedin and paclitaxel.

### BACKGROUND

Among traditional chemotherapeutic drugs, many of them are compounds, which have great limitations in clinical applications. Strategies to improve hydrophobic drugs include: designing prodrugs, which involves modifying the structure of compounds to increase the water solubility, but usually results in reduced activity; and using emulsification techniques, which involves dispersing hydrophobic drugs into oil phases, but usually results in instability problems. With the emergence of nanotechnology, amphiphilic block copolymers are used to encapsulate drugs in the hydrophobic cores of micelles, etc. Micelles are formed spontaneously by amphiphilic substances in an aqueous solution upon reaching a certain concentration (critical micelle concentration) through intermolecular forces such as hydrogen bonding, van der Waals forces, and electrostatic interactions. The micelles have a core-shell structure consisting of a hydrophobic core and a hydrophilic shell. Unlike the aqueous environment inside liposomes, the core of micelles provides a hydrophobic environment, making it highly compatible with hydrophobic drugs—a principle known as “like dissolves like”. In addition, the micelles also have characteristics such as structural stability, pH sensitivity, and mucoadhesion. However, since most polymer molecules are usually exogenous substances having large molecular weights, they can easily elicit immune responses upon entering the body. Therefore, while increasing drug solubility, the safety of materials should also be considered to reduce carrier toxicity. PCL-PEOz is an amphiphilic polymeric material having good long-circulating characteristics in vivo and good biocompatibility. PCL, as a hydrophobic block, has crystallinity and biocompatibility; because its melting temperature is around 60° C., it readily forms crystals with drugs at room temperature, thereby forming a more stable hydrophobic core, which improves drug loading and the stability of the micelles during in vivo circulation. PEOz, as a hydrophilic block, not only imparts flexibility and prevents protein adsorption, but also exhibits pH sensitivity; when the pH value is less than 6.5, micelles containing PEOz as the hydrophilic block are prone to disassembly, thereby effectively releasing drugs. Since the pH in endosomes of tumor cells is about 5.4, and when the environmental pH is lower than the pka of PEOz, the tertiary amide groups within the molecules make the linked oxygen atoms susceptible to protonation, resulting in PEOz being positively charged, thereby generating electrostatic repulsion between chains, which causes the structure of nanoparticles to become loose, followed by rapid drug release, avoiding degradation by lysosomes. In addition, compared with PEG, PEOz is more controllable in synthesis, more readily allows modification of terminal functional groups by chemical reactions to link different targeting groups, and is more favorable for industrialization.

Paclitaxel, due to its extremely poor water solubility and complex structure, is difficult to be stably encapsulated in common materials. The drug may leak prematurely in the blood circulation and cause certain toxic side effects after non-specific binding to tissue. Despite being a current research focus, liposomes still suffer from defects such as low drug loading capacity and instability. Trabectedin is a novel hydrophobic antitumor drug isolated and extracted from the Caribbean tunicate Ecteinascidia turbinata, effective against multiple malignant tumors, and exhibits strong anticancer activity at low concentrations (nM). Unlike the traditional antitumor drug paclitaxel, which works by inhibiting tubulin formation, trabectedin is structurally classified as a tetrahydroisoquinoline natural product; the tetrahydroisoquinoline ring of trabectedin can bind into the minor groove of DNA and generate interactions, thereby inducing DNA double-strand breaks and leading to cell death. Besides directly killing tumor cells, studies show that trabectedin can selectively deplete monocytes and macrophages in the blood and spleen of tumor-bearing mice, inhibit the transition of macrophages to the tumor-promoting M2 phenotype, and increase the numbers of CD8⁺ and CD4⁺ tumor-infiltrating T cells in tumors, thereby restoring tumor immunity. Currently, the clinical injection formation of trabectedin (Yondelis®) is prepared by mixing sucrose, potassium dihydrogen phosphate, and trabectedin in a mass ratio of 400:27.2:1, and has a pH range of 3.6-4.2. Yondelis® fails to protect trabectedin during the blood circulation, resulting in high toxicity and thereby limiting further clinical application.

### SUMMARY

To overcome the problems existing in the prior art, such as the limitations in in vivo delivery technology for combinations of hydrophobic drugs and antibodies, an object of the present disclosure is to provide a nanopreparation suitable for loading trabectedin and paclitaxel; in particular, to provide a nanopreparation of hydrophobic drugs such as paclitaxel and trabectedin, and on this basis, to link an antibody to the drug-loaded nanoparticle to form an antibody-nanoparticle complex.

In other words, an object of the present disclosure is to provide a drug delivery system capable of tightly encapsulating hydrophobic drugs to form a stable and uniform nanopreparation, then linking an antibody to the surface of the nanopreparation, and ultimately achieving antibody-linked long-acting circulation in vivo and targeted delivery to specific cells. More specifically, in the present disclosure, an amphiphilic crystalline block copolymer is used to encapsulate hydrophobic drugs to construct a non-targeted nanopreparation, and an antibody is linked to the non-targeted drug-loaded nanoparticle to form an antibody-nanoparticle targeted nanopreparation. Although in the examples of the present disclosure, anti-EGFR monoclonal antibody cetuximab and anti-HER2 monoclonal antibody are respectively used to link to poly(ε-caprolactone)-poly(2-ethyl-2-oxazoline) (PCL-PEOz-COOH) loaded with hydrophobic chemical drugs to form efficient and safe nanocrystalline micelle systems that target specific cell surfaces (targeted nanocrystalline micelles), the present disclosure is suitable for antibody-nanoparticle complexes containing other antibodies and crystalline amphiphilic block copolymers loaded with hydrophobic chemical drugs.

In the present disclosure, PCL-PEOz is used to encapsulate paclitaxel and trabectedin, that is, the hydrophobic chemical drugs can be tightly encapsulated in PCL-PEOz using the easily crystallizable property of poly(ε-caprolactone); using the characteristic that a terminus of poly(2-ethyl-2-oxazoline) is readily linkable to different functional groups, the purposes of adjusting the delivery system for different tumor targets and performing precise targeted delivery can be achieved; and the PEOz surface of the micelle carrier can achieve long circulation and pH-sensitive functions of the carrier in vivo.

To achieve the above object and other related objects, the present disclosure provides the following technical solutions:

The present disclosure provides a targeted drug-loaded micelle loaded with paclitaxel, including a non-targeted drug-loaded micelle loaded with paclitaxel and a first targeting group linked to a first micelle carrier; the non-targeted drug-loaded micelle has a first shell-core structure, and the micelle first carrier is prepared from poly(ε-caprolactone)-poly(2-ethyl-2-oxazoline) (PCL-PEOz), where a hydrophobic block PCL and paclitaxel jointly form a core of the first shell-core structure, and a hydrophilic block PEOz forms a shell of the first shell-core structure; the first targeting group is linked via an amide bond formed between —NH₂ on a surface of the first targeting group and —COOH at a terminus of the PEOz on a surface of the shell.

As an embodiment of the present disclosure, in the non-targeted drug-loaded micelle loaded with paclitaxel, the paclitaxel is at a drug loading capacity of 2% to 10%.

As an embodiment of the present disclosure, the first targeting group is selected from the group consisting of a targeting antibody and a targeting peptide; the targeting antibody includes a full-length monoclonal antibody or a fragment of an antibody having targeting functionality.

As an embodiment of the present disclosure, the first targeting group includes cetuximab; a molar ratio of —COOH at the terminus of the PEOz to —NH₂ on the surface of the first targeting group is in a range of (0.1-10):1.

As an embodiment of the present disclosure, the targeted drug-loaded micelle loaded with paclitaxel further includes hyaluronic acid; a molar ratio of —NH₂ on the surface of the first targeting group to —COOH on a surface of hyaluronic acid is in a range of 1:(1-20).

The present disclosure further provides a method for preparing the targeted drug-loaded micelle loaded with paclitaxel as mentioned above, the method including the following steps:
A1. mixing the PCL-PEOz-COOH for preparing the first micelle carrier with paclitaxel to obtain a mixture, co-dissolving the mixture in an organic solvent to obtain a mixed material, and adding water to the mixed material to form a mixed solution of the organic solvent and the water, alternatively subjecting the mixed material to ultrasonic treatment to form a homogeneous emulsion;
A2. removing the organic solvent from the mixed solution or the homogeneous emulsion by rotary evaporation under reduced pressure or rotary evaporation under vacuum, and removing an unencapsulated drug (by microporous membrane filtration) to obtain the non-targeted drug-loaded micelle;
A3. blending the non-targeted drug-loaded micelle with the first targeting group, adding a catalyst selected from the group consisting of 4-(4,6-dimethoxytriazin-2-yl)-4-methylmorpholine hydrochloride (DMTMM) and a mixture of 1,3-dicyclohexylcarbodiimide (DCC) and N-hydroxysuccinimide (NHS), and performing a chemical reaction to obtain the targeted drug-loaded micelle loaded with paclitaxel.

In step A1, the organic solvent is selected from the group consisting of chloroform, and a volume ratio of chloroform to water is in a range of 1:(5-10). In another embodiment, the organic solvent is selected from the group consisting of tetrahydrofuran and methanol, and a volume ratio of the organic solvent to water is in a range of 1:(1-30).

The present disclosure further provides use of the targeted drug-loaded micelle loaded with paclitaxel as mentioned above in the preparation of a combination preparation of paclitaxel and trabectedin.

As an embodiment of the present disclosure, in the combination preparation, the trabectedin is in a form of a targeted drug-loaded micelle loaded with trabectedin.

As an embodiment of the present disclosure, the targeted drug-loaded micelle loaded with trabectedin includes a non-targeted drug-loaded micelle loaded with trabectedin and a second targeting group linked to a second micelle carrier; the second micelle carrier is prepared from a material selected from the group consisting of poly(ε-caprolactone)-polyethylene glycol (PCL-PEG), poly(ε-caprolactone)-poly(2-ethyl-2-oxazoline) (PCL-PEOz), poly(lactic-co-glycolic acid)-polyethylene glycol (PLGA-PEG), and poly(lactic-co-glycolic acid)-poly(2-ethyl-2-oxazoline) (PLGA-PEOz), and the non-targeted drug-loaded micelle loaded with trabectedin has a second shell-core structure, where a hydrophobic block PCL or PLGA and trabectedin jointly form a core of the second shell-core structure, and a hydrophilic block PEG or PEOz forms a shell of the second shell-core structure; the second targeting group is linked via an amide bond formed between —NH₂ on a surface of the second targeting group and —COOH at a terminus of the PEG or the PEOz on a surface of the shell.

As an embodiment of the present disclosure, in the non-targeted drug-loaded micelle loaded with trabectedin, the trabectedin is at a drug loading capacity of 2% to 10%.

As an embodiment of the present disclosure, the second targeting group is selected from the group consisting of a targeting antibody and a targeting peptide; the targeting antibody includes a full-length monoclonal antibody or a fragment of an antibody having targeting functionality.

As an embodiment of the present disclosure, the second targeting group is a peptide selected from the group consisting of CSPGAK (Cysteine-Serine-Proline-Glycine-Alanine-Lysine, also known as mUNO) that specifically targets tumor-associated macrophages; a molar ratio of —COOH at the terminus of the PEG or the PEOz to —NH₂ on the surface of the second targeting group is in a range of (0.1-10):1.

The present disclosure further provides a method for preparing the targeted drug-loaded micelle loaded with trabectedin as mentioned above, the method including the following steps:
B1. mixing the second micelle carrier selected from the group consisting of PCL-PEG, PCL-PEOz-COOH, PLGA-PEG and PLGA-PEOz with trabectedin to obtain a mixture, co-dissolving the mixture in an organic solvent, and adding water to form a mixed solution of the organic solvent and the water, alternatively subjecting the mixed solution to ultrasonic treatment to form a homogeneous emulsion;
B2. removing the organic solvent from the mixed solution or the homogeneous emulsion by rotary evaporation under reduced pressure or rotary evaporation under vacuum, and removing an unencapsulated drug (by microporous membrane filtration) to obtain a non-targeted drug-loaded micelle; and
B3. blending the non-targeted drug-loaded micelle with the second targeting group, adding a catalyst selected from group consisting of the 4-(4,6-dimethoxytriazin-2-yl)-4-methylmorpholine hydrochloride (DMTMM) and a mixture of 1,3-dicyclohexylcarbodiimide (DCC) and N-hydroxysuccinimide (NHS), and performing a chemical reaction to obtain the targeted drug-loaded micelle loaded with trabectedin.

In step B1, the organic solvent is selected from the group consisting of chloroform, and a volume ratio of chloroform to water is 1:(5-10). In another embodiment, the organic solvent is selected from the group consisting of tetrahydrofuran and methanol, and a volume ratio of the organic solvent to water is 1:(1-30).

(1) In an embodiment, as an example, paclitaxel is used as a hydrophobic chemical drug model. However, the present disclosure is not limited to this specific hydrophobic chemical drug.

In some embodiments, the second targeting group is a targeting antibody. The targeting antibody includes at least one selected from the group consisting of a full-length monoclonal antibody and a fragment of an antibody having targeting functionality.

In an embodiment, as an example, trastuzumab is used as the second targeting group. However, the present disclosure is not limited to this specific second targeting group.

The PCL-PEOz is an amphiphilic block copolymer formed from a hydrophobic block poly(ε-caprolactone) (PCL) and a hydrophilic block poly(2-ethyl-2-oxazoline) (PEOz).

In an embodiment, in PCL-PEOz, the PCL has a molecular weight of 2000 Da-5000 Da; and the PEOz has a molecular weight of 2000 Da.

The PCL-PEOz-COOH is an amphiphilic block copolymer bearing carboxyl groups, which is formed from a hydrophobic block poly(ε-caprolactone) (PCL) and a hydrophilic block poly(2-ethyl-2-oxazoline) (PEOz).

In an embodiment, in the targeted drug-loaded micelle, a mass ratio of PCL5000-PEOz2000-COOH) to PCL2000-PEOz2000-COOH) is (10 to 1):1.

In an embodiment, a mass of the hydrophobic chemical drug is 2% to 10% of a total mass of the PCL5000-PEOz2000-COOH) and the PCL2000-PEOz2000-COOH).

In an embodiment, the second targeting group is linked via an amide bond formed between —NH₂ on the surface of the second targeting group and —COOH at the terminus of the PEOz by an acylation reaction.

In an embodiment, the catalyst used in the acylation reaction is selected from the group consisting of 4-(4,6-dimethoxytriazin-2-yl)-4-methylmorpholine hydrochloride (DMTMM) and a mixture of 1,3-dicyclohexylcarbodiimide (DCC) and N-hydroxysuccinimide (NHS).

In an embodiment, —NH₂ on the surface of the second targeting group which is not reacted with —COOH at the terminus of the PEOz forms an amide bond with —COOH on the surface of hyaluronic acid by an acylation reaction.

(2) In an embodiment, as an example, trabectedin is used as a hydrophobic chemical drug model. However, the present disclosure is not limited to this specific hydrophobic chemical drug.

In some embodiments, the second targeting group is selected from the group consisting of a targeting peptide and a targeting antibody.

In an embodiment, as an example, peptide mUNO (CSPGAK) is used as the second targeting group. However, the present disclosure is not limited to this specific second targeting group.

The PCL-PEOz is an amphiphilic block copolymer formed from a hydrophobic block poly(ε-caprolactone) (PCL) and a hydrophilic block poly(2-ethyl-2-oxazoline) (PEOz).

In an embodiment, in PCL-PEOz, the PCL has a molecular weight of 2000 Da-5000 Da; and the PEOz has a molecular weight of 2000 Da.

The PCL-PEOz-COOH is an amphiphilic block copolymer bearing carboxyl groups, which is formed from a hydrophobic block poly(ε-caprolactone) (PCL) and a hydrophilic block poly(2-ethyl-2-oxazoline) (PEOz).

In an embodiment, in the targeted drug-loaded micelle, a mass ratio of PCL₅ₖ-PEOz₂ₖ-COOH to PCL2K-PEOz₂ₖ-COOH is (10 to 1):1.

In an embodiment, a mass of the hydrophobic chemical drug is 2% to 12% of a total mass of PCL₅ₖ-PEOz₂ₖ-COOH and PCL₂ₖ-PEOz₂ₖ-COOH.

In an embodiment, the second targeting group is linked via an amide bond formed between —NH₂ on the surface of the second targeting group and —COOH at a terminus of PEOz by an acylation reaction.

In an embodiment, the catalyst used in the acylation reaction is 4-(4,6-dimethoxytriazin-2-yl)-4-methylmorpholine hydrochloride (DMTMM).

In the prior art, the primary approach involves using the nanotechnology to form micelles from PCL-PEG loaded with the hydrophobic drug paclitaxel; however, due to the difficulty in linking functional groups to a terminus of PEG, there have been no reports on targeted micelles. There are very few reports on the delivery of trabectedin using nanotechnology, besides the single clinical product Yondelis®, which is formed by mixing sucrose, potassium dihydrogen phosphate, and trabectedin in a mass ratio of 400:27.2:1. Trabectedin has a specific killing effect against tumor-associated macrophages. In the present disclosure, PCL-PEOz is used to respectively encapsulate paclitaxel and trabectedin to form crystalline micelles, which not only solves the problem of in vivo delivery of both drugs but also addresses the problem of safe, efficient, and targeted delivery of trabectedin that Yondelis® has failed to solve. The innovation of the present disclosure lies not only in achieving effective in vivo delivery of paclitaxel and trabectedin but also in realizing comprehensive targeted delivery of paclitaxel to different subtypes of tumor cells by linking antibodies and hyaluronic acid that targets CD44 overexpressed on tumor cell surfaces. This is combined with targeted delivery of trabectedin targeting tumor-associated macrophages, achieving a synergistic therapy of targeted tumor therapy and immunotherapy and obtaining better therapeutic efficacy than single-agent therapy (see FIGS. 14 and 15).

Compared with the prior art, some embodiments of the present disclosure have the following beneficial effects:
(1) in the present disclosure, hydrophobic chemical drugs can be tightly encapsulated in micelles using the easily crystallizable property of poly(ε-caprolactone);
(2) the PEOz surface of the micelle carrier of the present disclosure can achieve long circulation and pH-sensitive functions of the carrier in vivo;
(3) the targeting group linked to the micelle carrier of the present disclosure can specifically target specific cell surfaces, and the micelle enters the cells via receptor-mediated endocytosis, thereby increasing drug uptake.

As described above, in the present disclosure, the hydrophobic chemical drugs can be tightly encapsulated in the micelles using the easily crystallizable property of poly(ε-caprolactone), thereby ensuring the stability of the drugs before reaching target cells; and the micelles formed from an amphiphilic polymer block copolymer have a particle size of approximately 100 nm, and this particle size, together with the hydrophilic PEOz shell, helps the micelles avoid recognition by the reticuloendothelial system, thereby possessing a long-circulation function. In experiments, it is found that the combination of trabectedin nanopreparation encapsulated using PCL-PEOz and oxaliplatin results in higher tumor inhibition effect and reduced toxicity compared to using oxaliplatin alone.

### BRIEF DESCRIPTION OF THE DRAWINGS

By reading the following detailed description made with reference to the drawings for non-limiting examples, the other features, objects and advantages of the present disclosure will become more apparent:

FIG. 1A shows in vitro drug release profiles of PTX@PCL₅ₖ-PEOZ₅ₖ in Example 3 of the present disclosure, FIG. 1B shows in vitro drug release profiles of PTX@PCL₂ₖ-PEOZ₂ₖ in Example 3 of the present disclosure, and FIG. 1C shows in vitro drug release profiles of PTX@PCL-PEOz (1:1) in Example 3 of the present disclosure;

FIG. 2A shows particle size and polydispersity Index (PDI) changes of PTX@PCL-PEOz (1:1) in PBS and 50% FBS in Example 4 of the present disclosure, FIG. 2B shows particle size and polydispersity Index (PDI) changes of PTX@PCL₅ₖ-PEOZ₂ₖ in PBS and 50% FBS in Example 4 of the present disclosure, and FIG. 2C shows particle size and polydispersity Index (PDI) changes of PTX@PCL₂ₖ-PEOz₂ₖ in PBS and 50% FBS in Example 4 of the present disclosure;

FIG. 3A shows transmission electron micrograph of PTX@PCL₂ₖ-PEOZ₂ₖ in Example 5 of the present disclosure, FIG. 3B shows transmission electron micrograph of PTX@PCL-PEOz (1:1) in Example 5 of the present disclosure, and FIG. 3C shows transmission electron micrograph of PTX@PCL₅ₖ-PEOZ₅ₖ in Example 5 of the present disclosure;

FIG. 4 shows a schematic diagram of the reaction of the targeted micelle loaded with paclitaxel and the targeted micelle loaded with trabectedin in Example 6 and Example 14 of the present disclosure;

FIG. 5A shows time-of-flight mass spectrum of blank micelle in Example 7 of the present disclosure, FIG. 5B shows time-of-flight mass spectrum of trastuzumab in Example 7 of the present disclosure, FIG. 5C shows time-of-flight mass spectrum of blank micelle+trastuzumab in Example 7 of the present disclosure, FIG. 5D shows time-of-flight mass spectrum of trastuzumab+DMTMM in Example 7 of the present disclosure, and FIG. 5E shows time-of-flight mass spectrum of micelle+trastuzumab+DMTMM in Example 7 of the present disclosure;

FIG. 6 shows cellular uptake of the targeted micelles and the non-targeted micelles in Example 8 of the present disclosure;

FIG. 7A shows endocytic pathways via HER2 receptor of the targeted micelles in cells in Example 9 of the present disclosure, and FIG. 7B shows endocytic pathways via CD44 receptor of the targeted micelles in cells in Example 9 of the present disclosure;

FIG. 8A shows cytotoxicity of the micelles loaded with paclitaxel on SKBR-3 cells in Example 10 of the present disclosure, and FIG. 8B shows cytotoxicity of the micelles loaded with paclitaxel on SKOV-3 cells in Example 10 of the present disclosure;

FIG. 9A shows size of the micelle loaded with trabectedin in Example 12 of the present disclosure, and FIG. 9B shows PDI of the micelle loaded with trabectedin in Example 12 of the present disclosure;

FIG. 10 shows cytotoxicity of the micelle loaded with trabectedin on ovarian cancer cells in Example 16 of the present disclosure;

FIG. 11 shows cytotoxicity of the micelle loaded with trabectedin on a co-culture system of ovarian cancer cells and macrophages in Example 16 of the present disclosure;

FIG. 12 shows cytotoxicity of the combination of the micelle loaded with paclitaxel and the micelle loaded with trabectedin on a co-culture system of ovarian cancer cells and macrophages in Example 17 of the present disclosure;

FIG. 13A shows activity of the micelle loaded with trabectedin on organoids derived from sample 1 in Example 18 of the present disclosure, and FIG. 13B shows activity of the micelle loaded with trabectedin on organoids derived from sample 2 in Example 18 of the present disclosure;

FIG. 14 shows tumor volume change of the mice in each group within 31 days after intravenous administration in tumor-bearing mice (sample 1) in Example 19 of the present disclosure;

FIG. 15 shows tumor volume change of the mice in each group within 31 days after intravenous administration in tumor-bearing mice (sample 2) in Example 19 of the present disclosure;

FIG. 16 shows body weight change of the mice in each group within 31 days after intravenous administration in tumor-bearing mice (sample 1) in Example 19 of the present disclosure;

FIG. 17 shows body weight change of the mice in each group within 31 days after intravenous administration in tumor-bearing mice (sample 2) in Example 19 of the present disclosure;

FIG. 18A shows change in the number of peripheral blood neutrophils in the mice after the end of intravenous administration in tumor-bearing mice (sample 1) in Example 19 of the present disclosure, and FIG. 18B shows change in the number of peripheral blood neutrophils in the mice after the end of intravenous administration in tumor-bearing mice (sample 2) in Example 19 of the present disclosure;

FIG. 19A shows change in the number of peripheral blood Ly6Chi monocytes in the mice after the end of intravenous administration in tumor-bearing mice (sample 1) in Example 19 of the present disclosure, and FIG. 19B shows change in the number of peripheral blood Ly6Chi monocytes in the mice after the end of intravenous administration in tumor-bearing mice (sample 2) in Example 19 of the present disclosure.

### DETAILED DESCRIPTION OF THE EMBODIMENTS

The present disclosure will be described in detail below with reference to examples. The following examples will help a person skilled in the art to further understand the present disclosure, but do not limit the present disclosure in any way. It should be noted that a person of ordinary skill in the art could also make several variations and improvements without departing from the spirit of the present disclosure. These variations and improvements should all fall within the scope of the present disclosure.

### 1. Experimental Reagents and Sources of Cells, Tissues, and Animals

PCL₅ₖ-PEOZ₂ₖ-COOH and PCL₂ₖ-PEOz₂ₖ-COOH were both purchased from Xi'an RuixiBiotech Co., Ltd., China; paclitaxel was purchased from TiTan Technology (Shanghai) Co. Ltd., China; TAXOL was supplied by Bristol-Myers Squibb; Herceptin (trastuzumab) was sourced from Genetech (South San Francisco, CA, USA); peptide mUNO (CSPGAK) was purchased from QYAOBIO; anti-CD44 antibody was purchased from Abcam; trabectedin was sourced from Zhejiang Zhongkechuangyue Pharmaceutical Co., Ltd., China; chloroform (analytical grade), absolute ethanol (analytical grade), acetonitrile (chromatographic grade), and dimethyl sulfoxide (analytical grade) were purchased from Sinopharm Chemical Reagent Co., Ltd., China; 4-(4,6-dimethoxytriazin-2-yl)-4-methylmorpholine hydrochloride (DMTMM) was purchased from Shanghai Adamas Reagent Co., Ltd., China; phosphotungstic acid was purchased from Shanghai Macklin Biochemical Technology Co., Ltd., China; RPMI-1640, DMEM high-glucose medium, McCOY's medium, L-15 medium, 0.25% pancreatin, penicillin-streptomycin mixed solution (dual antibiotics, 100×), and fetal bovine serum (FBS) were purchased from Gibco, USA; CCK-8 cell proliferation and cytotoxicity assay kit was purchased from Beyotime; dialysis bag (10000 MW) was purchased from Thermo Fisher Scientific (China) Co., Ltd.; and Oxaliplatin for Injection (AoBo) was purchased from Nanjing Pharmaceutical Factory Co., Ltd., China.

SKBR-3, RAW264.7, ID-8, and SKOV-3 were purchased from Shanghai Fuheng Biotechnology Co., Ltd., China; B-NDG mice were purchased from Biocytogen Co., Ltd; and experimental ovarian cancer samples were provided by the Shanghai First People's Hospital, China, with patients informed consent and in compliance with ethical regulations.

### 2. Experimental Instruments

Rotary evaporator: Shanghai Luyi Industry & Trade Co., Ltd., China; water bath sonicator: Kunshan Ultrasonic Instruments Co., Ltd., China; laser scattering particle size analyzer: Malvern Panalytical, UK; biological transmission electron microscope: FEI; confocal fluorescence microscope: Leica, Germany; microplate reader; flow cytometer: BD company, USA; CO2 constant temperature incubator: Thermo Fisher Scientific, USA. Centrifuge 5418R: Eppendorf; ultra-clean workbench, 4° C. refrigerator: Haier Group Corporation, China; BS 210S electronic balance: Sartorius; high-performance liquid chromatograph (HPLC), chromatographic column ZORBAX SB-C18 (5 μm, 4.6×150 mm): Agilent Technologies, Co., Ltd.

### Example 1. Preparation of the Micelle Loaded with Paclitaxel

### 1. Preparation Process:

In PCL-PEOz₂ₖ-COOH used in this example, the molecular weight of PCL was 2000-5000 Da; and the molecular weight of PEOz was 2000 Da.

Specifically, the method for preparing the micelle loaded with paclitaxel was conducted as follows:

Preparation of three types of high molecular polymer stock solutions (5 mg/mL): the materials used for micelles, i.e., PCL₅ₖ-PEOz₂ₖ-COOH, PCL₅ₖ-PEOz₂ₖ-COOH and 40 mg of PCL₂ₖ-PEOz₂ₖ-COOH (1:1), and PCL₂ₖ-PEOz₂ₖ-COOH, were respectively prepared as 5 mg/mL tetrahydrofuran stock solutions and stored at 4° C.

(1) Preparation of the Micelle Loaded with Paclitaxel by THE Dialysis

Preparation of paclitaxel stock solution (2.5 mg/mL): 40 mg of paclitaxel powder was accurately weighed into a glass bottle, 16 mL of tetrahydrofuran was added and complete dissolution and uniform mixing were conducted to obtain a mixture, and the mixture was sealed by using a pressure-sensitive adhesive, and stored at 4° C.

Specific preparation method: Preparation of three types of micelles loaded with paclitaxel: 160-600 μL (200 μL was selected in this example) of PTX THE stock solution and 1 mL of the above high molecular polymer THE stock solution (5 mg of polymer) were drawn into a 50 mL round bottom flask by using a microsyringe, about 4 mL of THF was supplemented to obtain a mixture, and the mixture was uniformly shaken. 2 mL of deionized water was added dropwise to induce micellization under vigorous stirring at 1000 rpm. Most of the THF was slowly removed by rotary evaporation under vacuum conditions in a water bath at 18° C., and the resulting solution was then transferred into a dialysis bag (molecular weight cut-off (MWCO)=10 KDa) for static dialysis overnight in 2000 mL of deionized water. Then, the residual THF was removed by stirred dialysis in deionized water and phosphate buffered saline (PBS). Finally, the micelle solution was filtered through a 0.45 μm microporous filter membrane to remove unencapsulated PTX, and the final product was stored at 4° C. for later use.

As can be seen from Table 1, for the three types of micelles loaded with paclitaxel prepared by THE dialysis, the particle sizes are all below 200 nm, the PDIs are all less than 0.3, and the particle size of the micelles increases with the increasing proportion of the PCL₅ₖ block in the material.

TABLE 1
Particle size and PDI of blank micelles
and micelles loaded with paclitaxel

| | Material | Particle | |
|---|---|---|---|
| Loaded drug | ratio ^{a} | size (nm) | PDI |
| Blank micelle | 1:1 | 85.0 ± 10.54 | 0.141 ± 0.09 |
| Blank micelle | 0:1 | 142.2 ± 9.30 | 0.133 ± 0.07 |
| Paclitaxel | 1:0 | 66.8 ± 6.21 | 0.172 ± 0.04 |
| Paclitaxel | 1:1 | 73.1 ± 7.89 | 0.244 ± 0.04 |
| Paclitaxel | 0:1 | 158.5 ± 11.43 | 0.201 ± 0.08 |

^{a} Mass ratio of PCL₅ₖ-PEOZ₂ₖ-COOH to PCL₂ₖ-PEOZ₂ₖ-COOH

(2) Preparation of the Micelle Loaded with Paclitaxel by Emulsification-Evaporation

Preparation of three types of high molecular polymer stock solutions (5 mg/mL):80 mg of a polymer material was accurately weighed into a glass bottle (the stock solution for PTX@PCL₅ₖ-PEOZ₂ₖ micelle was 80 mg of PCL₅ₖ-PEOz₂ₖ-COOH, the stock solution used for PTX@PCL-PEOz (1:1) micelle was 40 mg of PCL₅ₖ-PEOz₂ₖ-COOH and 40 mg of PCL₂ₖ-PEOz₂ₖ-COOH, and the stock solution used for PTX@PCL₂ₖ-PEOZ₂ₖ micelle was 80 mg of PCL₂ₖ-PEOz₂ₖ-COOH), 16 mL of chloroform was added and complete dissolution and uniform mixing were conducted to obtain a mixture, and the mixture was sealed with a pressure-sensitive adhesive, and stored at 4° C.

Preparation of paclitaxel stock solution (2.5 mg/mL):40 mg of paclitaxel powder was accurately weighed into a glass bottle, 16 mL of chloroform was added and complete dissolution and uniform mixing were conducted to obtain a mixture, and the mixture was sealed with a pressure-sensitive adhesive, and stored at 4° C.

Specific preparation method: Preparation of three types of micelles loaded with paclitaxel: A specified amount of the stock solution of PCL₅ₖ-PEOz₂ₖ-COOH or PCL₂ₖ-PEOz₂ₖ-COOH or a mixture of PCL₅ₖ-PEOz₂ₖ-COOH and PCL₂ₖ-PEOz₂ₖ-COOH was drawn into a round bottom flask by using a microsyringe, and a specified amount of paclitaxel stock solution was added, such that the mass ratio of paclitaxel to (paclitaxel and material) was 2% to 15% (8% was selected in this example). A specified amount of deionized water was added, such that the volume ratio of chloroform to deionized water in the system was 1:5 to 1:10 (1:10 was selected in this example). The resulting system was completely mixed, and subjected to sonication in a water bath at 100% power until a milky white, uniform emulsion was formed. Chloroform was removed by rotary evaporation under vacuum conditions at 37° C. to obtain a non-targeted micelle solution. Finally, the micelle solution was filtered through a 0.45 μm microporous filter membrane to remove unencapsulated PTX, and the final product was stored at 4° C. for later use.

As can be seen from Table 2, for the three types of micelles loaded with paclitaxel prepared by emulsification-evaporation, the particle sizes are all below 200 nm, the PDIs are all less than 0.3.

TABLE 2
Particle size and PDI of blank micelles
and micelles loaded with paclitaxel

| | Material | Particle | |
|---|---|---|---|
| Loaded drug | ratio ^{a} | size (nm) | PDI |
| Blank micelle | 1:1 | 133.2 ± 2.3 | 0.233 ± 0.01 |
| Blank micelle | 0:1 | 129.3 ± 1.1 | 0.254 ± 0.01 |
| Paclitaxel | 1:0 | 139.6 ± 1.82 | 0.239 ± 0.01 |
| Paclitaxel | 1:1 | 127.3 ± 2.83 | 0.205 ± 0.01 |
| Paclitaxel | 0:1 | 148.6 ± 1.3 | 0.232 ± 0.01 |

^{a} Mass ratio of PCL₅ₖ-PEOZ₂ₖ-COOH to PCL₂ₖ-PEOZ₂ₖ-COOH

### Example 2. Determination of Drug Loading Capacity of the Micelle Loaded with Paclitaxel

To investigate the encapsulation efficiency and actual drug loading capacity of the three types of non-targeted micelles loaded with paclitaxel prepared by THE dialysis in Example 1, the following liquid chromatography conditions were established for investigation: chromatographic column: ZORBAX SB-C18 (5 μm, 4.6×150 mm), mobile phase: acetonitrile:water (50:50, V/V), column temperature: 30° C., flow rate 1 mL/min, injection volume: 10 μL, detection wavelength: 227 nm. The specific process was conducted as follows: an appropriate amount of the lyophilized powder of the drug-loaded micelle was accurately weighed, completely dissolved in acetonitrile by vortexing, then filtered through a 0.22 μm microporous membrane, and analyzed by HPLC injection. The actual paclitaxel content was calculated by substituting into the standard curve, and the drug loading capacities of PTX@PCL₂ₖ-PEOz₂ₖ, PTX@PCL-PEOz (1:1), and PTX@PCL₅ₖ-PEOZ₂ₖ micelles, calculated by substituting into the following formula, were 5.3±0.6(%), 6.7±0.6(%), and 8.6±0.2(%), respectively.

$$Encapsulation efficiency ( \% ) = Mass paclitaxel in purified micelle / Mass of paclitaxel input * 100$$

$$Drug loading capacity ( \% ) = Mass of paclitaxel in micelle / Total mass of drug - loaded micelle * 100.$$

### Example 3. In Vitro Release Determination of the Micelle Loaded with Paclitaxel

The method for detecting release from the micelle loaded with paclitaxel prepared by THF dialysis in Example 1 was conducted as follows: dynamic membrane dialysis was used and performed at 37° C. Phosphate buffered saline (PBS) at different pH values (pH 7.4, pH 6.5, and pH 5.4) was used to simulate pH conditions in the blood circulation in vivo, tumor microenvironment, and lysosomes, respectively. Tween-80 (1%, w/w) was added to the PBS release medium as a solubilizer to meet sink conditions. A release medium at the corresponding pH was added to the PCL-PEOz micelle loaded with paclitaxel to a volume of 3 mL (containing 250 μg of PTX). The resulting mixture was then transferred into a dialysis bag (MWCO=10 KDa). The dialysis bag was then completely immersed in 15 mL of release medium at 37° C. In vitro release experiments were performed at 37° C. and 100 rpm. At predetermined time points, 200 μL of the release medium was withdrawn for further determination, and an equal volume of pre-warmed fresh release medium was replenished. The withdrawn 200 μL of release medium was lyophilized, then reconstituted by using 200 μL of acetonitrile through sonication for 30 minutes, then filtered through a 0.22 μm microporous filter membrane, and subsequently detected by HPLC injection. Each point was determined in triplicate, and the average peak area was substituted into the HPLC standard curve to calculate the paclitaxel content.

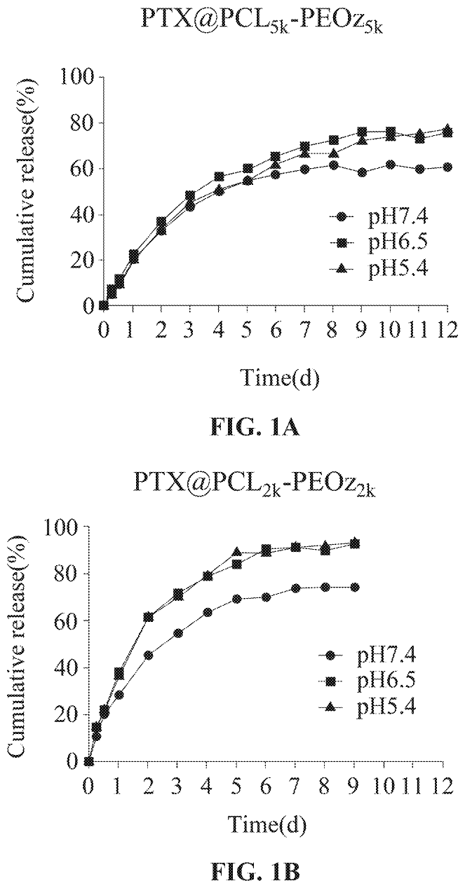

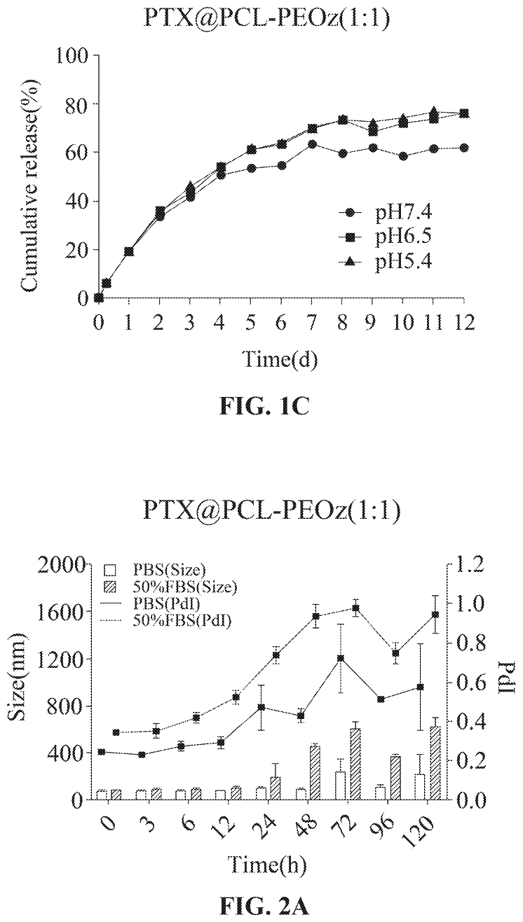

As can be seen from FIGS. 1A to 1C, PTX@PCL₅ₖ-PEOZ₂ₖ and PTX@PCL-PEOz (1:1) exhibit similar release behaviors, where the cumulative release at day 1 is only 20%, under pH 6.5 and pH 5.4 conditions, the cumulative release reaches 75% around day 9, and under pH 7.4 conditions, the cumulative release is 60%; and for the PTX@PCL₂ₖ-PEOZ₂ₖ drug-loaded micelle, under pH 6.5 and pH 5.4 conditions, the cumulative release at day 1 is 40%, which is twice that of PTX@PCLs-PEOZ₂ₖ and PTX@PCL-PEOz (1:1) under the same conditions, the cumulative release around day 6 reaches 90%, and under pH 7.4 conditions, the cumulative release is 75%. The average release rate under pH 6.5 conditions is higher than that under pH 7.4 conditions due to certain acid sensitivity of PCL-PEOz. In the tumor microenvironment, where the pH is weakly acidic, the preparation can promote drug release at the tumor site, thereby enhancing the targeting effect and reducing systemic toxic side effects of the drug.

### Example 4. Serum Stability Determination of the Micelle Loaded with Paclitaxel

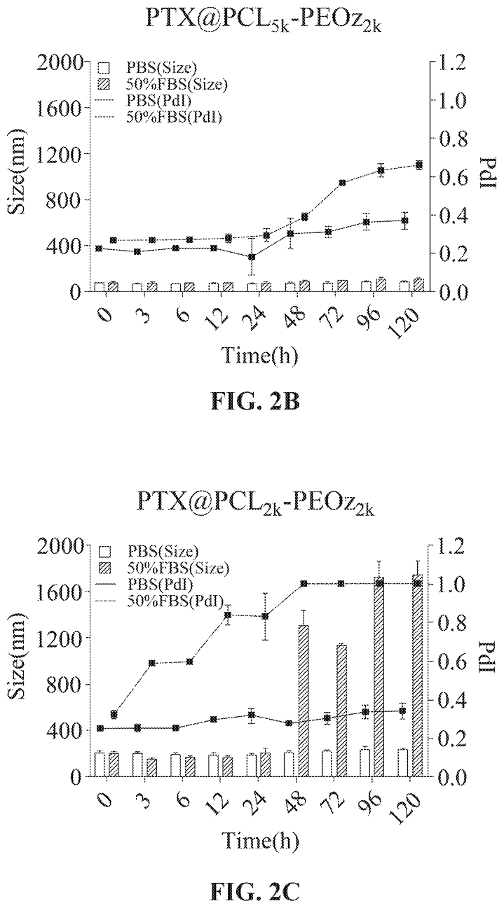

Using particle size and polydispersity index as indicators, the stability of the non-targeted micelle loaded with paclitaxel prepared by THE dialysis in Example 1 within three days in serum was detected to mimic the in vivo environment. Specifically, the micelle solution and serum were uniformly mixed at a 1:1 volume ratio and placed in a constant temperature incubator at 37° C. The particle size and PDI of the mixed solvent were determined at specific time points, respectively. The results are shown in FIGS. 2A to 2C. In the PBS solution at 37° C., the particle size and PDI of the micelles of the three carriers remained substantially unchanged within 72 h; and in the 50% FBS solution at 37° C., the particle size and PDI of PTX@PCL₅ₖ-PEOZ₂ₖ and PTX@PCL-PEOz (1:1) remained substantially unchanged within 48 hours, while PTX@PCL₂ₖ-PEOz₂ₖ remained stable within 24 hours, indicating good stability of the present carrier.

### Example 5. Morphology Observation of the Micelle Loaded with Paclitaxel

Transmission electron microscopy was used to morphologically observe the previously prepared blank micelle and the three types of non-targeted drug-loaded micelles prepared by THE dialysis in Example 1. The specific method was conducted as follows: deionized water was used as the dispersion medium to dilute the above micelles to 0.5 to 1 mg/ml (carrier concentration). Then, 10 μL of the diluted micelles was dropped onto a copper grid coated with a carbon film. After 1 minute, excess micelle solution was blotted dry with filter paper. Subsequently, 10 μL of phosphotungstic acid was dropped onto the copper grid to counterstain the micelles on the grid. After 1 minute, the solution was blotted dry with filter paper. After standing overnight, the samples were observed using a biological transmission electron microscope.

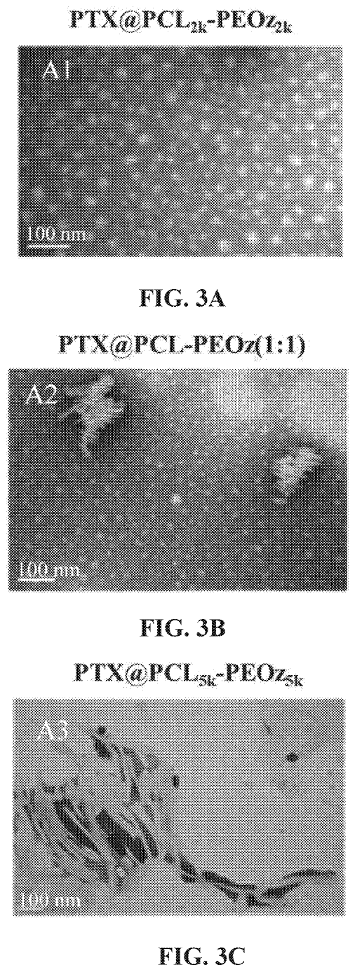

The determination results are shown in FIGS. 3A to 3C. It can be seen that the morphologies of the micelles prepared from the three carriers are different, where among the micelles loaded with paclitaxel, the PTX@PCL₂ₖ-PEOZ₂ₖ micelle is spherical, the PTX@PCL-PEOz (1:1) micelle includes both spherical and rod-shaped forms, and the PTX@PCL₅ₖ-PEOZ₂ₖ micelle is rod-shaped.

### Example 6. Preparation of Targeted the Micelle Loaded with Paclitaxel

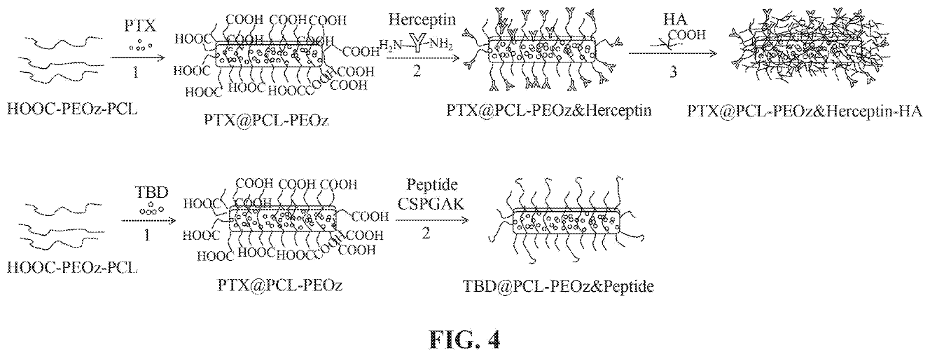

The targeted drug-loaded micelle was prepared via an amidation reaction. Specifically, in a phosphate buffered saline solution, the carboxyl groups on the surface of the non-targeted drug-loaded micelle (PTX@PCL₅ₖ-PEOZ₂ₖ) prepared by emulsification-evaporation in Example 1 was reacted with the primary amino groups on the surface of antibody trastuzumab under the action of DMTMM to form stable amide bonds, where a molar ratio of —COOH to —NH₂ in the reaction was (10 to 0.1):1 (0.1:1 was selected in this example), and the reaction was performed at 2° C.-10° C. (4° C. was selected in this example) and 350 rpm for 24 hours. Then, a resulting system was further reacted with hyaluronic acid, where a molar ratio of —NH₂ to —COOH in the reaction was 1:(1-20) (1:20 was selected in this example), to form the targeted micelle loaded with paclitaxel (FIG. 4). The resulting targeted drug-loaded micelle was stored at 4° C. for later use.

The preparation process for the blank targeted micelle was the same as that for the targeted micelle loaded with paclitaxel.

### Example 7. Verification of the Linking Between the Antibody and the Micelle in the Targeted Micelle Loaded with Paclitaxel

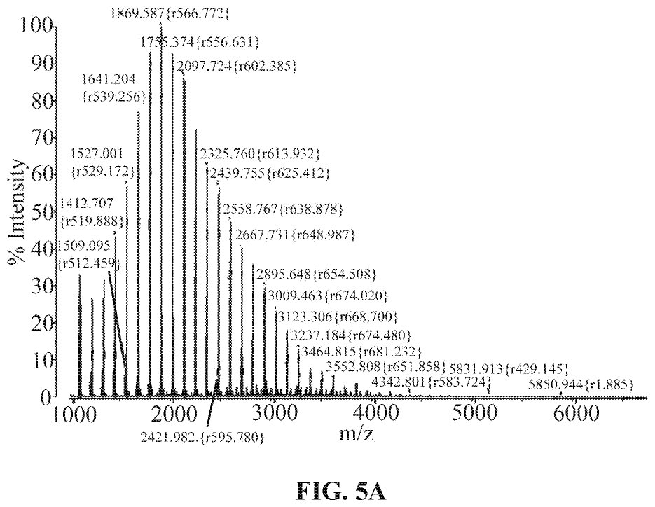

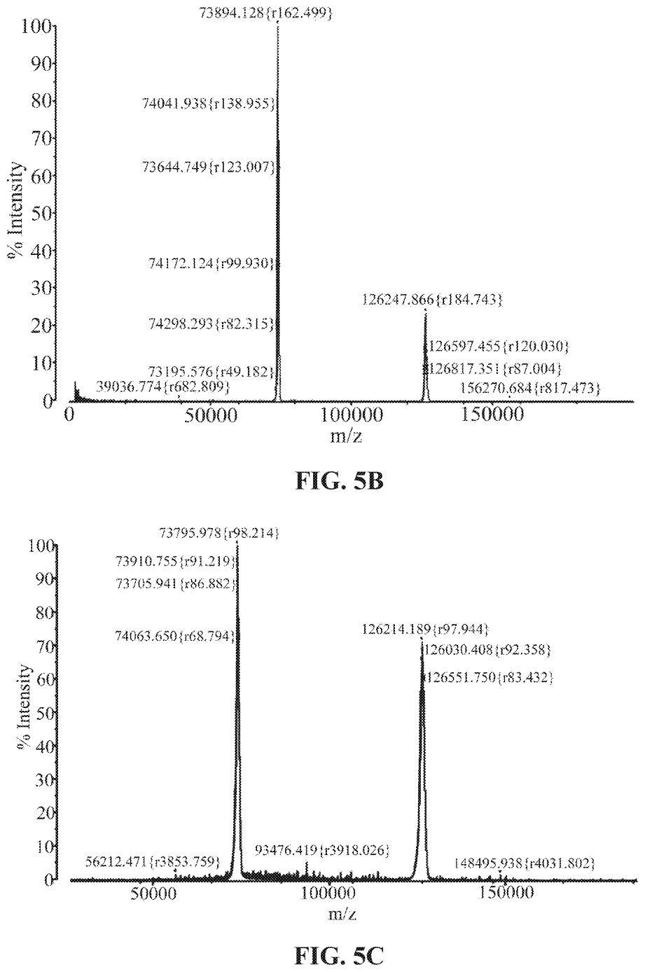

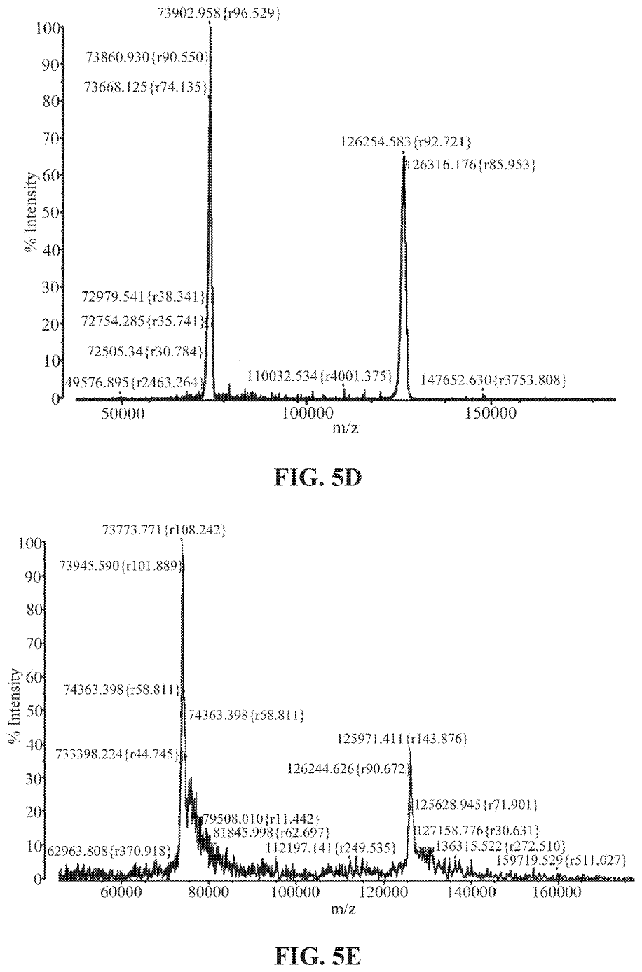

Matrix-assisted laser desorption/ionization time-of-flight mass spectrometry (MALDI-TOF-MS) was used to verify the successful preparation of the targeted drug-loaded micelle. MALDI-TOF-MS works as follows: a macromolecular sample is mixed with a large amount of matrix; the matrix absorbs laser light and transfers energy to the sample, generating molecular ions. This avoids direct laser irradiation of the analyte, providing an ideal method for analyzing thermally labile biological macromolecules and polymers, and offers high sampling rates and sensitivity. The experimental method was conducted as follows: Five samples were prepared respectively at a concentration of 1-2 mg/mL (1 mg/mL was selected in this example): blank micelle, antibody, mixture of blank micelle and antibody, mixture of antibody and DMTMM, and targeted micelle (mixture of micelle+antibody+DMTMM). Specifically, these were: the PCL-PEOz (1:1) blank micelle prepared in Example 1, trastuzumab, mixture of PCL-PEOz (1:1) blank micelle and trastuzumab, mixture of trastuzumab and DMTMM, and mixture of PTX@PCL-PEOz (1:1) micelle+trastuzumab+DMTMM. All five samples were reacted under targeted micelle preparation conditions, and then sent to the detection center for detection. FIG. 5A shows that the distribution of ion peaks approximates a normal distribution, with a difference of 114 between adjacent ion peaks, corresponding to one polymerization unit of PCL. FIG. 5B shows that the molecular weight of trastuzumab is approximately 185 kD, and in view of FIG. 5C, FIG. 5D, and FIG. 5E, the NMR peak of the targeted micelle changes from the original single peak to a set of peaks exhibiting a normal distribution. Furthermore, a decrease in ion peak signal intensity appears at the highest intensity position (m/z=1400-2500) in the normally-distributed-like spectrum of the micelle, verifying the linkage between the polymer block at this location and Herceptin. It is thus concluded that the antibody is successfully linked to the micelle.

### Example 8. Investigation of Cellular Uptake of the Targeted Micelles and the Non-Targeted Micelles

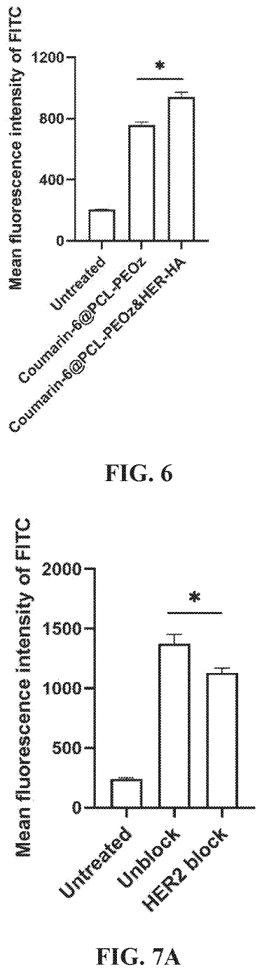

Coumarin-6 was used instead of paclitaxel to prepare a non-targeted micelle loaded with coumarin-6 (the preparation method was the same as emulsification-evaporation procedure described in Example 1, the drug loading capacity was 0.1%, and the material used was PCL₅ₖ-PEOz₂ₖ-COOH) and a targeted micelle loaded with coumarin-6 (the preparation method was the same as in Example 6). The ovarian cancer cell line SKOV-3 with high expressions of HER2 and CD44 was used. After trypsin digestion and counting, the cells were added to a 12-well plate at 100000 cells per well, with three replicate wells per group. Culturing was further conducted for 12 hours under 5% CO₂ conditions, then the original medium was removed, and 1 mL of serum-free DMEM medium was added, such that the final concentration of coumarin-6 in each well was 100 ng/ml. Incubation was then conducted at 37° C. for 2 hours. After incubation, the cells were washed three times by using cold PBS. The cells in each group were digested with trypsin, centrifuged at 4° C. and 1000 rpm for 3 minutes, the supernatant was discarded, and the cells were resuspended in 1 mL of PBS. This process was repeated three times. Finally, the cells were resuspended in 0.5 mL of PBS in a flow cytometry tube for detection. As can be seen from FIG. 6, the uptake of the targeted micelle by SKOV-3 is significantly higher than that of the non-targeted micelle, indicating that the linking of the antibody and hyaluronic acid on the micelle can target receptors on the cell surface so as to increase the cellular uptake of the micelle.

### Example 9: Investigation of Endocytic Pathways of the Targeted Micelles in Cells

The endocytosis of the targeted micelle via the HER2 receptor pathway and the CD44 receptor pathway was investigated separately. The specific method was conducted as follows: Coumarin-6 was used instead of paclitaxel to prepare a targeted micelle loaded with coumarin-6 (the preparation method was the same as in Example 6). The ovarian cancer cell line SKOV-3 with high expressions of HER2 and CD44 was used. After trypsin digestion and counting, the cells were added to a 12-well plate at 100000 cells per well, with three replicate wells per group.

The endocytosis of the targeted micelle via the HER2 receptor pathway was investigated, specifically as follows: 1 mg/mL Herceptin was added to the unblock group. Incubation was conducted at 37° C. for 40 minutes, and then the targeted micelle loaded with coumarin-6 was respectively added to the unblock group and the block group, such that the final concentration of coumarin-6 was 100 ng/mL. Another incubation was conducted at 37° C. for 2 hours, and then the cells were washed three times by using cold PBS. The cells in each group were digested with trypsin, centrifuged at 4° C. and 1000 rpm for 3 minutes, the supernatant was discarded, and the cells were resuspended in 1 ml of PBS. This process was repeated three times. Finally, the cells were resuspended in 0.5 ml of PBS in a flow cytometry tube for detection.

The endocytosis of the targeted micelle via the CD44 receptor pathway was investigated, specifically as follows: 16 μg/mL anti-CD44 antibody was added to the unblock group. Incubation was conducted at 37° C. for 30 minutes, and then the targeted micelle loaded with coumarin-6 was respectively added to the unblock group and the block group, such that the final concentration of coumarin-6 was 100 ng/mL. Another incubation was conducted at 4° C. for 1 hour, and then the cells were washed three times by using cold PBS. The cells in each group were digested with trypsin, centrifuged at 4° C. and 1000 rpm for 3 minutes, the supernatant was discarded, and the cells were resuspended in 1 ml of PBS. This process was repeated three times. Finally, the cells were resuspended in 0.5 ml of PBS in a flow cytometry tube for detection.

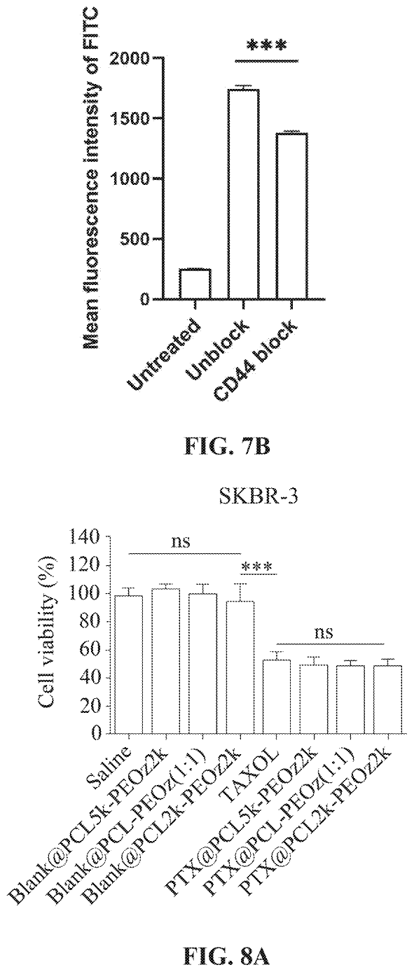

As can be seen from FIGS. 7A to 7B, after blocking the HER2 and CD44 antibodies on the cells using the corresponding antibodies, the cellular uptake of the targeted micelle decreases significantly, indicating that the targeted micelle can increase endocytosis by targeting the HER2 and CD44 receptors on the cell surface via Herceptin and hyaluronic acid, respectively.

### Example 10. Investigation of Cytotoxicity of the Micelle Loaded with Paclitaxel on Cells

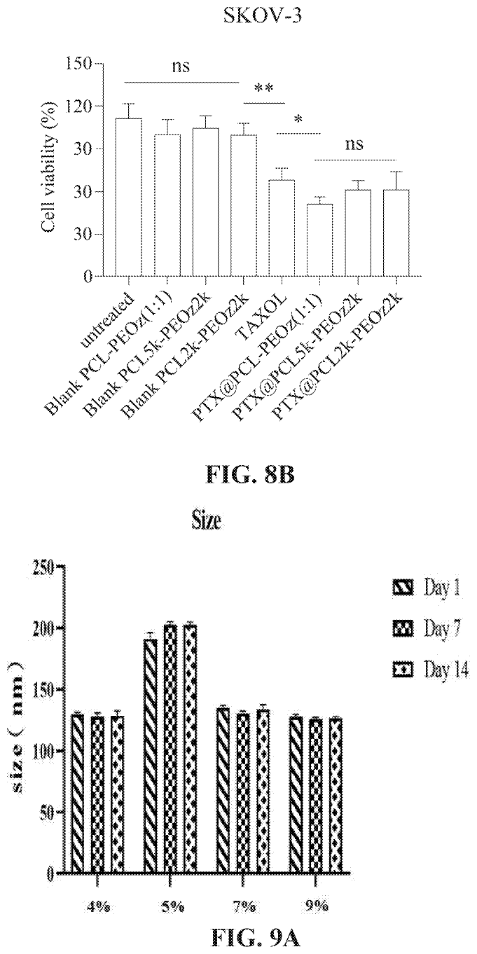

SKBR-3 and SKOV-3 cells in the logarithmic growth phase were selected and plated in 96-well plates at 8000 cells per well, with three replicate wells per group. Culture was conducted for 24 hours, and then the medium was removed. 100 μL of fresh medium containing different non-targeted drug-loaded micelles (PTX@PCL₅ₖ-PEOZ₂ₖ, PTX@PCL-PEOz (1:1), or PTX@PCL₂ₖ-PEOZ₂ₖ) was respectively added to administration groups, such that the final concentration of paclitaxel in each well was 100 ng/ml; The micelle at the same concentration as the micelle concentration in the administration groups was added to the blank micelle groups (PCL₅ₖ-PEOZ₂ₖ, PCL-PEOz (1:1), and PCL₂ₖ-PEOZ₂ₖ). Incubation was conducted for 48 hours, and then detection was performed using CCK-8 assay. Specifically, 10 μL of CCK-8 reagent was added to each well, and incubated at 37° C. for 30 minutes, and the absorbance was determined at 450 nm using a microplate reader. As can be seen from FIGS. 8A to 8B, the blank micelles show no cytotoxicity, indicating that the carriers used have a good safety; and the three types of drug-loaded micelles all show significant cytotoxicity against SKBR-3 and SKOV-3 cells, and no differences are shown among the three types of drug-loaded micelles.

### Example 11. Preparation of the Micelle Loaded with Trabectedin

In PCL₅ₖ-PEOZ₂ₖ-COOH used in this example, the molecular weight of PCL was 5000 Da; and the molecular weight of PEOz was 2000 Da.

Specifically, the method for preparing a micelle loaded with trabectedin was conducted by the following steps:

Preparation of PCL₅ₖ-PEOZ₂ₖ-COOH stock solution: 20 mg of polymer material was accurately weighed into a glass bottle, and 1 mL of chloroform was added to obtain a polymer material stock solution with a concentration of 20 mg/mL.

Preparation of trabectedin stock solution (2.5 mg/mL):1.0 mg of trabectedin powder was accurately weighed into a glass bottle, and 1 mL of chloroform was added to obtain a trabectedin stock solution with a concentration of 1 mg/mL.

Preparation method: A specified amount of the PCL₅ₖ-PEOz₂ₖ-COOH stock solution was drawn into a round bottom flask by using a microsyringe, and a specified amount of trabectedin stock solution was added, such that the mass ratio of trabectedin to (trabectedin and polymer material) was 2% to 10% (9% was selected in this example). Deionized water was slowly added at a rate of 0.9 to 4 mL/min (1 mL/min was selected in this example), such that the volume ratio of chloroform to deionized water in the system was 1:5 to 1:12 (1:10 was selected in this example). The system was completely mixed, and sonicated in a water bath at 100% power until a milky white, uniform emulsion was formed. Chloroform was removed by rotary evaporation under vacuum conditions at 37° C. to obtain the trabectedin micelle solution.

### Example 12. Stability Determination of the Micelle Loaded with Trabectedin

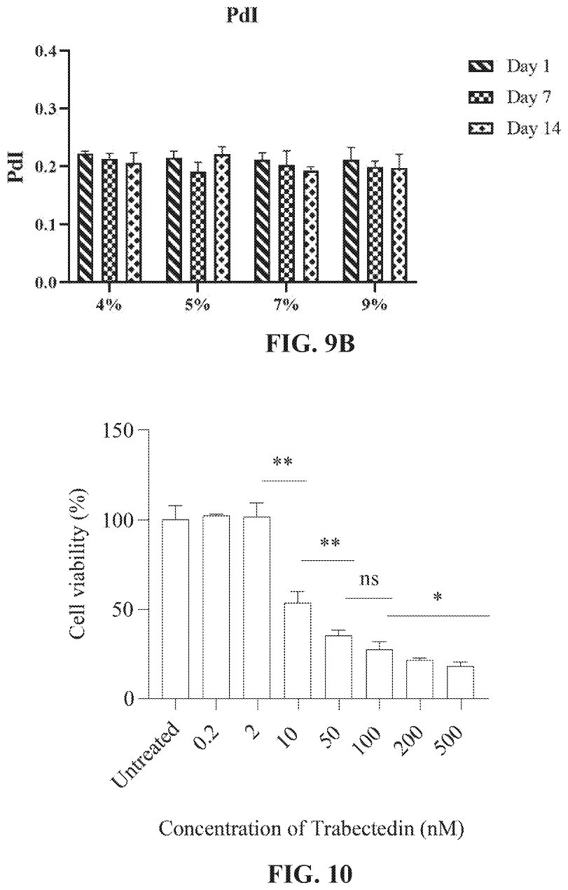

Using particle size and polydispersity index as indicators, the stability of the non-targeted drug-loaded micelle in Example 10 within 14 days at 4° C. was detected to investigate the storage stability of the preparation. Specifically, the targeted micelle sample was stored in a refrigerator at 4° C., and the particle size and PDI of the sample were determined at specific time points. The results are shown in FIGS. 9A to 9B. It can be seen that within 14 days, the particle size of the targeted micelle shows no significant changes, and the PDI remains below 0.3, indicating that the targeted micelle has good storage stability under 4° C. conditions.

### Example 13. Determination of Drug Loading Capacity of the Micelle Loaded with Trabectedin

To investigate the encapsulation efficiency and actual drug loading capacity of the trabectedin micelle in Example 11, the following liquid chromatography conditions were established for investigation: chromatographic column: Shim-pack GIST (5 μm, 4.6×250 mm), mobile phase: aqueous potassium dihydrogen phosphate solution methanol (gradient elution), column temperature: 40° C., flow rate: 0.8 ml/min, detection wavelength: 210 nm. The specific process was conducted as follows: a trabectedin micelle was prepared using emulsification-evaporation with a drug input of 9%; unencapsulated trabectedin was removed by ultrafiltration; the micelle was lyophilized, then an appropriate amount of the lyophilized powder of the drug-loaded micelle was accurately weighed, completely dissolved in acetonitrile by vortexing, and centrifuged at 13200 rpm for 30 minutes; and the supernatant was taken and subjected to HPLC analysis. The actual trabectedin content was calculated by substituting into the standard curve, and the drug loading capacity and encapsulation efficiency of the trabectedin micelle, calculated by substituting into the following formulas, were 2.6% and 28.9%, respectively.

$$Encapsulation efficiency ( \% ) = Mass of trabectedin in purified micelle / Mass of trabectedin input * 100$$

$$Drug loading capacity ( \% ) = Mass of trabectedin in micelle / Total m ass of drug - loaded micelle * 100.$$

### Example 14. Preparation of the Targeted Micelle Loaded with Trabectedin

The targeted drug-loaded micelle was prepared via an amidation reaction. Specifically, in a phosphate buffered saline solution, the carboxyl groups on the surface of the non-targeted drug-loaded micelle in Example 10 and the primary amino groups on the surface of peptide mUNO (CSPGAK), which targets receptor CD206 overexpressed on the surface of macrophages, formed stable amide bonds under the action of DMTMM, where a —COOH/—NH₂ molar ratio=(10 to 0.1):1 (1:1 was selected in this example), and the reaction was performed at 2° C.-10° C. (4° C. was selected in this example) and 350 rpm for 24 hours. The resulting targeted drug-loaded micelle was stored at 4° C. for later use (FIG. 4).

### Example 15. Verification of the Linking Between the Peptide and the Micelle

Elemental analysis was used to verify the linking between peptide CSPGAK and micelle. Specifically, 20 mg of PCL₅ₖ-PEOZ₂ₖ-COOH was taken to prepare a blank micelle, and the blank micelle was directly mixed with 0.804 mg of peptide. 20 mg of PCL₅ₖ-PEOZ₂ₖ-COOH was taken to prepare a targeted micelle (the preparation method was the same as in Examples 11 and 14), such that the molar ratio of carboxyl groups on the material to amino groups on peptide CSPGAK was (10 to 0.1):1 (1:1 was selected in this example). The two groups of micelles were placed in dialysis bags (MWCO=10 kDa), and dialyzed against 2 L of ultrapure water at 4° C. to remove the peptide that was not linked to the micelle, with water changed every 1 hour for a total of five water changes. The samples were dislyzed and then lyophilized. The PCL₅ₖ-PEOz₂ₖ-COOH material and the two groups of lyophilized micelles were sent to the detection center for detection using an elemental analyzer. The PCL-PEOz-COOH material contained no S element, while the peptide mUNO contained S element; therefore, the S content in each group was used to verify the linking between peptide and micelle. As shown in Table 3, the S element content in the PCL-PEOz-COOH is less than 0.1%, approximately considered as containing no S; the S element content in the PCL-PEOz blank micelle and mUNO mixture group is 0.14%, and the S element content in the targeted micelle group is 0.28%. The results verify the linking between peptide and micelle.

TABLE 3
Characterization of the linking between peptide mUNO and micelle

| Group | S content (%) |
|---|---|
| PCL-PEOz group | <0.1 |
| PCL-PEOz blank micelle and mUNO mixture group | 0.14 ± 0.02 |
| PCL-PEOz-mUNO targeted micelle group | 0.28 ± 0.07 |

### Example 16. Investigation of Cytotoxicity of the Micelle Loaded with Trabectedin on Cells

The cytotoxicity of the non-targeted micelle loaded with trabectedin in Example 11 on ovarian cancer cells SKOV-3 was investigated. The specific method was conducted as follows; SKOV-3 cells in the logarithmic growth phase were selected and plated in 96-well plates at 5000 cells per well, with three replicate wells per group. Culture was conducted for 24 hours, then the medium was removed, and 100 μL of fresh medium containing trabectedin micelle was added, such that the final concentration of the trabectedin drug was 0.2 nM-500 nM. Incubation was conducted for 48 hours, and detection was performed. Specifically, 10 μL of CCK-8 reagent was added to each well, and incubated at 37° C. for 40 minutes, and the absorbance was determined at 450 nm using a microplate reader. As can be seen from FIG. 10, starting from 10 nM, trabectedin shows a significant killing effect on SKOV-3 cells.

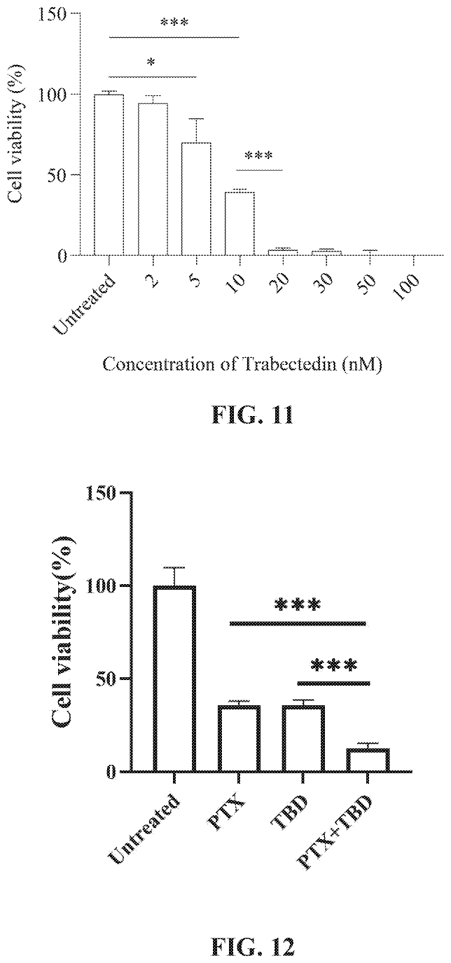

The cytotoxicity of the trabectedin micelle, i.e., the targeted micelle loaded with trabectedin in Example 13, on a co-culture system of ovarian cancer cells and macrophages was investigated. The specific method was conducted as follows: ID-8 cells and RAW264.7 cells in the logarithmic growth phase were selected and plated in 96-well plates at 5000 cells per well, with three replicate wells per group, and a ratio of ID-8 cells to RAW264.7 cells in each well of 1:1. Culture was conducted for 24 hours, then the medium was removed, and 100 μL of fresh medium containing trabectedin micelle was added, such that the final concentration of the trabectedin drug was 2 nM-500 nM. Incubation was conducted for 48 hours, and detection was performed. Specifically, 10 μL of CCK-8 reagent was added to each well, and incubated at 37° C. for 40 minutes, and the absorbance was determined at 450 nm using a microplate reader. As can be seen from FIG. 11, trabectedin exhibits good killing efficacy against the co-culture system of ID-8 and RAW264.7, achieving complete killing when trabectedin reached 20 nM.

### Example 17. Investigation of Cytotoxicity of the Combination of the Micelle Loaded with Trabectedin and the Micelle Loaded with Paclitaxel

The cytotoxicity of the combination of the micelle loaded with trabectedin (the non-targeted micelle TBD@PCL₅ₖ-PEOz₂ₖ in Example 11) and the micelle loaded with paclitaxel (the non-targeted micelle PTX@PCL₅ₖ-PEOz₂ₖ prepared by emulsification-evaporation in Example 1) on a co-culture system of ovarian cancer cells and macrophages was investigated. The specific method was conducted as follows: ID-8 cells and RAW264.7 cells in the logarithmic growth phase were selected and plated in 96-well plates at 5000 cells per well, with three replicate wells per group, and a ratio of ID-8 cells to RAW264.7 cells in each well of 1:1. Culture was conducted for 24 hours, then the medium was removed, and 100 μL of fresh DMEM medium containing drug was added to each group, where the administration groups were respectively administered micelle loaded with trabectedin (trabectedin concentration: 15 nM), micelle loaded with paclitaxel (paclitaxel concentration: 642 nM), and combination of micelle loaded with trabectedin and micelle loaded with paclitaxel (trabectedin concentration: 7.5 nM, paclitaxel concentration: 321 nM). Incubation was conducted for 48 hours, and then detection was performed. Specifically, 10 μL of CCK-8 reagent was added to each well, and incubated at 37° C. for 40 minutes, and the absorbance was determined at 450 nm using a microplate reader. As can be seen from FIG. 12, the combination of trabectedin and paclitaxel shows a good killing effect on the co-culture system of ID-8 and RAW264.7, and the effect is superior to that of trabectedin or paclitaxel used alone.

### Example 18. In Vitro Activity of the Micelle Loaded with Trabectedin on Ovarian Cancer Organoid Model

### 1. Acquisition and Culture of Ovarian Cancer Primary Cells

Tumor primary cells were cultured using tumor tissue samples from different patients. The patient's pathological information was as follows:

Patient 1 (Source of Sample 1): Resected Specimen without Prior Chemotherapy

Right ovary: high-grade serous carcinoma, tumor size 7*5*3 cm, cancer emboli present in vasculature, no definite nerve invasion identified; cancer involvement observed on the serosal surface of the fallopian tube;

Immunohistochemistry results: (Slide G) Vim (few+), ER (+), PR (+), P53 (partial +), CK7 (partial +), Ki67 (+about 40%), PAX-2 (−), P16 (+), WT1 (−), WT1 (+), P16 (+), Vim (−); (Slide U) WT1 (−), PAX-8 (+), P53 (partial +), P16 (+); (Slide D′) WT1 (−), PAX-8 (+), P53 (weak+), P16 (+).

(Left adnexa) ovarian high-grade serous carcinoma, locally with squamous differentiation, cancer emboli present in vasculature, no definite nerve invasion identified; no cancer involvement found in the fallopian tube.

Immunohistochemistry results: Tumor cells: ER (majority +), PR (few+), P53 (+), CK7 (+), Ki67 (+about 70%), PAX-8 (partial +), WT1 (partial +), P16 (+), Calretinin (scant +), CK20 (−), Vim (−), Napsin-A (−).

Patient 2 (Source of Sample 2): Resected Specimen after 2 Cycles of Chemotherapy

Bilateral ovaries and fallopian tubes: high-grade serous carcinoma, involving the uterine serosal surface and subserosal layer.

Immunohistochemistry results: WT1 (+), P53 (+), ER (+), PR (partial +), PAX-8 (+), CK20 (−), CK7 (+), Ki67 (20%+), Napsin-A (−), Vim (−), P16 (+), GATA3 (−), α-Inhibin (−), P504s (−).

The method for extracting tumor primary cells was conducted as follows: After retrieval, the fresh tumor tissue sample was washed three times with a washing solution and minced into pieces with a size of 1 mm. Digestion solution (commercial digestion solution, composition: 1% to 3% penicillin/streptomycin, 0.05 to 0.2 mg/mL gentamicin, 0.3% to 0.8% nystatin, 1 to 4 mg/mL collagenase A, 0.05 to 0.1 mg/mL hyaluronidase, with Advanced DMEM/F12 making up the remainder) was added, followed by shaking incubation at 37° C. for 0.5 hours, with manual shaking every 5 min to ensure complete digestion. An equal volume of termination solution PBS (relative to the volume of digestion solution) was added to terminate digestion, followed by centrifugation at 1500 rpm, 4° C. for 5 min. The supernatant was discarded. Then, 2 mL of 2 mg/mL DNase I was added, followed by a water bath for 4 min to terminate digestion, centrifugation at 1500 rpm, 4° C. for 5 minutes, and the supernatant was discarded. The cell pellet was resuspended and filtered through a filter with a pore size of 100 μm to obtain a single-cell suspension, followed by centrifugation at 1500 rpm, 4° C. for 5 min. The supernatant was discarded. The cell pellet was subjected to red blood cell lysis using 1 mL of red blood cell lysis buffer, placed on ice for 3 minutes, then washed by adding three times the volume of washing solution, and centrifuged at 1500 rpm, 4° C. for 5 min. The supernatant was discarded, and the cells were resuspended. A medium (commercial medium, composition: 5 to 25 mM HEPES, 15 to 25 nM GlutaMAX, 1% to 3% B27, 50 to 200 ng/mL A83-01, 20 to 80 ng/mL EGF, 80 to 120 ng/ml Noggin, 300 to 600 ng/mL commercial R-spondin 1, 5 to 15 mM Y-27632, 0.5% to 2% P/S, 50 to 150 μg/mL Primocin, 200 to 300 nM SB202190, 10 to 15 ng/mL PGE2, 10 to 30 μg/mL FGF-7, 10 to 30 μg/mL FGF-10, basal medium: Advanced DMEM/F12) was added, followed by culture at 37° C., 5% CO2.

The method for constructing an ovarian cancer organoid model was conducted as follows: The tumor single-cell suspension was counted. At a cell density of 1× 10⁵ cells per well, the cell pellet was uniformly mixed with 15 μL of matrigel per well on ice, and seeded into the center of a 48-well plate, avoiding bubble generation. The 48-well plate was placed into a CO2 incubator and left to stand for 4 min. The matrigel was solidified, then the 48-well plate was taken out, and 300 μL of medium (commercial medium, composition as above) was added to each well, followed by culture in the CO2 incubator. Fresh medium was replenished every 3 days, and the organoids were passaged on day 12.

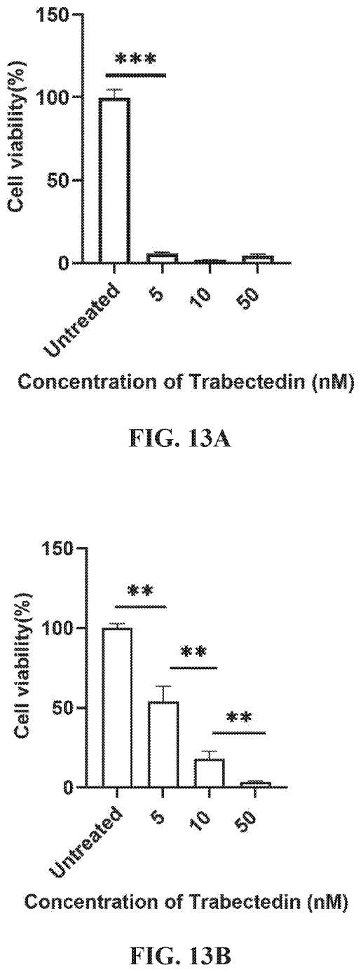

The cytotoxicity of the trabectedin preparation, i.e., the targeted micelle loaded with trabectedin in Example 13, on ovarian cancer organoids was investigated. The specific method was conducted as follows; Ovarian cancer organoids were taken and seeded in a 96-well plate at 5000 cells/well, with three replicate wells per group. 90 μL of medium was added, and culture was performed for 48 h. During administration, 10 μL of the targeted micelle loaded with trabectedin (TBD@PCL₅ₖ-PEOZ₂ₖ-mUNO) was added to each well, such that the final concentrations of the drug were 5 nM, 10 nM, and 50 nM. Incubation was performed for 48 hours, and then detection was performed using the ATP-TCA method. As can be seen from FIGS. 13A to 13B, the micelle loaded with trabectedin shows effective killing effects on organoid models derived from different ovarian cancer samples; furthermore, organoids derived from the chemotherapy-naive sample are more sensitive to trabectedin, indicating that using trabectedin for first chemotherapy may provide better benefit for patients.

### Example 19. In Vivo Pharmacodynamic Experiment in Mice

### 1. Establishment of Ovarian Cancer PDX Model

The PDX model was constructed using the tumor samples from Patient A and Patient B in Example 18, respectively. The method was conducted as follows: Female B-NDG mice (genotype: mut/mut, age: 7 weeks) were used, and the extraction of primary tumor cells was the same as in Example 16. After the primary tumor cells were expanded to a certain number, the cells were digested, and then resuspended in PBS to a final cell concentration of 10⁷ cells/mL. 100 μL of single-cell suspension was injected subcutaneously into the right back of each mouse. The needle hole was gently pressed for about 1 minute, and then the mouse was returned to the breeding cage. The formation of a small bulge under the skin indicated successful inoculation. The mice were observed weekly for general conditions such as mental state, diet, activity, and urination and defecation. Tumor formation under the skin was monitored. The period from the day of inoculation until the appearance of a palpable solid tumor mass at the inoculation site was defined as the latent period. The longest diameter (1) and the maximum transverse diameter in the vertical direction (w) of the tumor were recorded daily using a vernier caliper. The tumor volume was calculated according to the formula v=1*w*w/2. The tumor volume was calculated daily, and the tumor growth curve was plotted.

When the tumor volume in mice reached 1000-1200 mm³, the tumor was passaged. Specifically, the mice were euthanized by carbon dioxide asphyxiation, and the body surface was disinfected. Under sterile conditions, ovarian cancer tumor tissue samples were collected, cut into pieces with a size of 3 mm, and implanted subcutaneously near the axillary region of new mice to complete the passage. After the transplanted tumors were constructed, the mice were observed weekly for general conditions such as mental state, diet, activity, and urination and defecation. The longest diameter (1) and the maximum transverse diameter in the vertical direction (w) of the tumor were recorded daily using a vernier caliper. The tumor volume was calculated according to the formula v=1*w*w/2. The tumor volume was calculated daily, and the tumor growth curve was plotted. When the tumor in mice grew to 100-150 mm³, the pharmacodynamic experiments were performed.

### 2. In Vivo Pharmacodynamic Study in Mice

When the tumor in mice grew to 100-150 mm³, the mice were divided into eight groups, with 3 mice per group, ensuring that the average tumor volume in each group was similar. The eight groups of mice were as follows: PBS group, blank targeted micelle group (PCL₅ₖ-PEOZ₂ₖ-Herceptin-HA, blank micelle in Example 5), prepared PTX@PCL₅ₖ-PEOZ₂ₖ-Herceptin-HA group (prepared in Example 5), TBD@PCL₅ₖ-PEOZ₂ₖ-mUNO group (prepared in Example 13), PTX@PCL₅ₖ-PEOZ₂ₖ-Herceptin-HA+TBD@PCL₅ₖ-PEOZ₂ₖ-mUNO combination group, TAXOL group, carboplatin group, TAXOL+carboplatin combination group. The administration doses were paclitaxel 5 mg/kg, trabectedin 0.2 mg/kg, and carboplatin 50 mg/kg. The material concentration in the blank micelle group was the same as the maximum material concentration in the administration groups. Among them, the blank targeted micelle, PTX@PCL₅ₖ-PEOZ₂ₖ-Herceptin-HA, and TAXOL were administered once every two days, while TBD@PCL₅ₖ-PEOZ₂ₖ-mUNO and carboplatin were administered once every four days. When the tumor volume in the PBS group reached approximately 2000 mm³, administration was terminated. The body weight of the mice was determined every four days, and the tumor volumes were recorded.

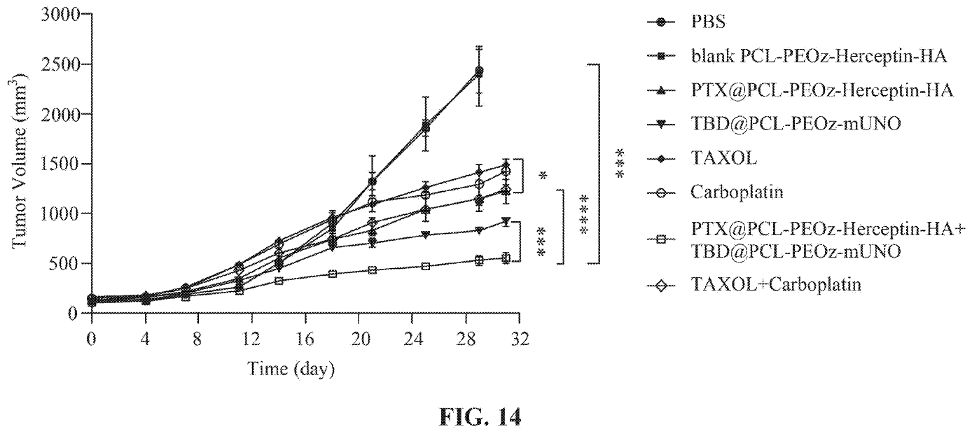

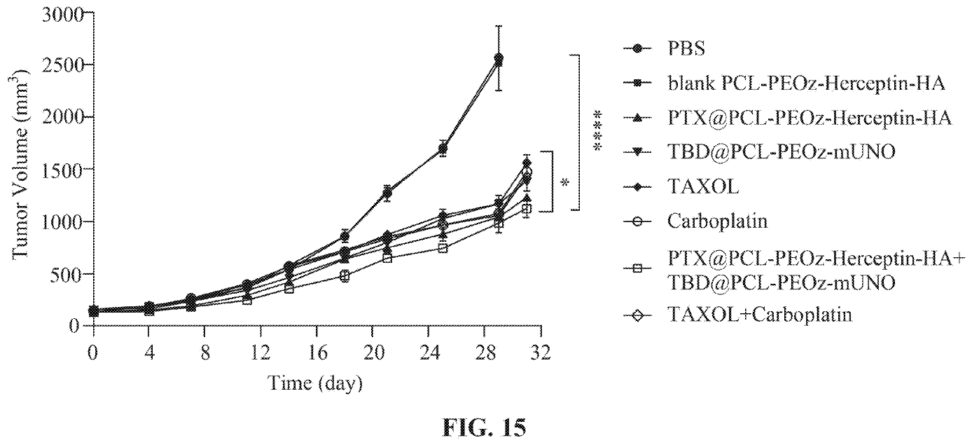

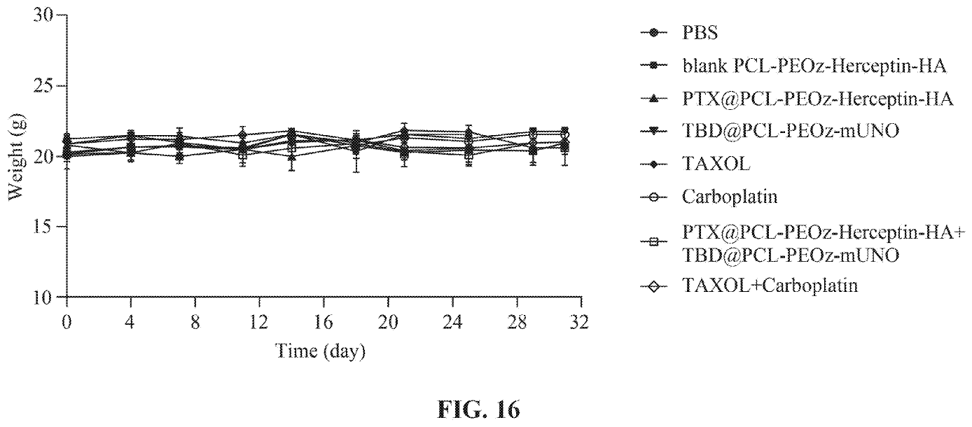

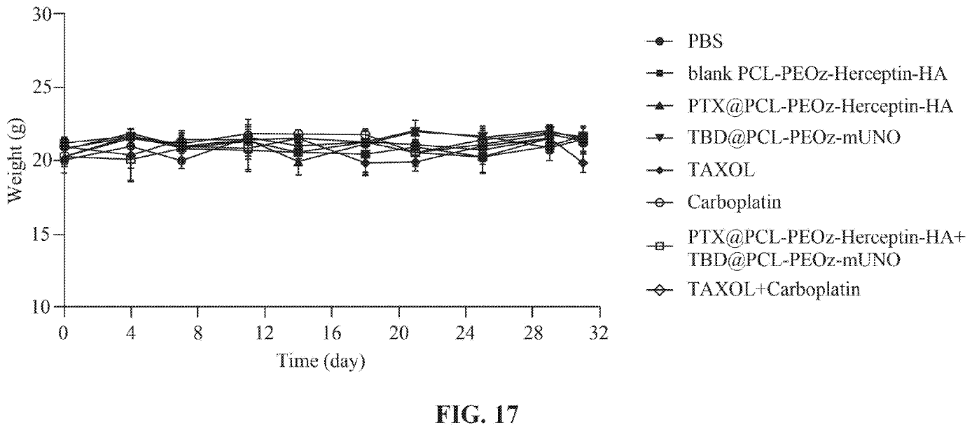

The results show that, as can be seen from the tumor growth curves in mice (FIG. 14, FIG. 15), within 31 days, the tumor volume in mice increases over time, and the models derived from the two samples exhibit similar growth trends after treatment. For sample 1 and sample 2, the tumor growth trend in the blank micelle (blank-PCL-PEOz-Herceptin-HA) group is the same as that in the PBS group, indicating that the blank targeted micelle has no effect on the tumor cells. Among them, the paclitaxel and trabectedin combination (PTX@PCL-PEOz-Herceptin-HA+TBD@PCL-PEOz-mUNO) group shows the best tumor inhibition effect, which is superior to the existing standard therapy TAXOL+carboplatin combination group. For sample 1, the tumor inhibition effect of the TBD@PCL-PEOz-mUNO group is superior to that of the other groups except the paclitaxel and trabectedin combination group, indicating that trabectedin has a good killing effect on ovarian cancer. For sample 2, the tumor inhibition effect of the paclitaxel and trabectedin combination group is superior to that of the TAXOL group, but no significant difference in antitumor effect is shown compared with the other drug treatment groups. This result corresponds to the experimental results at the in vitro organoid level, that is, patients without prior chemotherapy can obtain better benefits from trabectedin treatment. As can be seen from the body weight change curves in mice (FIG. 16, FIG. 17), the body weight of mice in each group shows no significant changes, indicating that the materials and the drugs in each group have good safety.

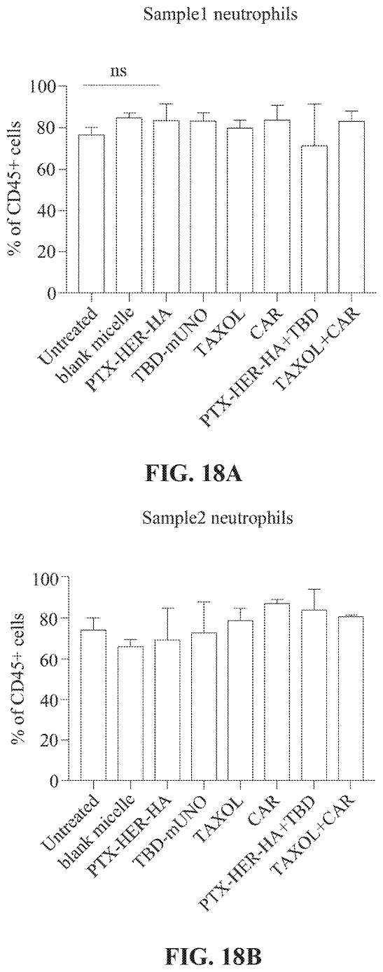

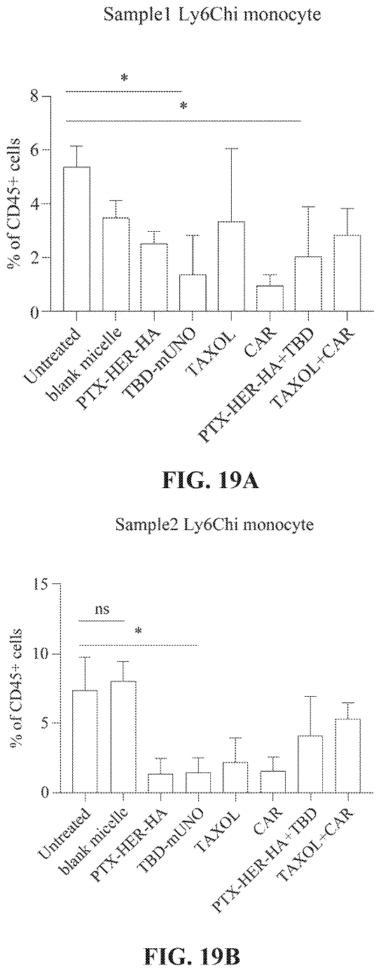

The peripheral blood monocytes in mice were analyzed. As can be seen from FIGS. 18A to 18B, after administration treatment in mice, the number of peripheral blood neutrophils does not change significantly, indicating that no systemic immune response or immunosuppression is induced in the mice after administration in each group. The number of neutrophils in mice in each group accounts for about 80% of the total white blood cells, which is higher than the 10%-25% observed in normal mice. This is due to the fact that the mice used are severely immunodeficient, with a lack of T and B cells, resulting in a relatively high proportion of neutrophils. As can be seen from FIGS. 19A to 19B, after administration treatment in mice, the proportion of Ly6Chi monocytes in the mice decreases, indicating that the drugs selectively deplete Ly6Chi monocytes in the mice. This result is consistent with literature reports that the number of Ly6Chi monocytes in the peripheral blood of mice decreases after trabectedin administration.

In summary, in the present disclosure, a pH-sensitive amphiphilic biodegradable material (such as PCL-PEOz) is used to encapsulate paclitaxel and trabectedin, respectively, to form stable micelles, thereby improving drug bioavailability, reducing the critical micelle concentration, and achieving long-acting circulation in vivo. By linking different antibodies and targeting groups to the surface of these micelles, the micelles can efficiently target tumor cells and tumor-associated macrophages, respectively, enter cells via receptor-mediated action, and release the drugs via self-degradation, thereby achieving combined targeted therapy with antibodies and chemical drugs for malignant tumors with high expression of specific antibody/antigen. In PDOX models, the combined therapy with the trabectedin and paclitaxel combination preparation is found to achieve a higher tumor inhibition effect than equivalent amounts of pure paclitaxel or trabectedin alone, and a 2.5-fold better tumor inhibition effect than carboplatin, while exhibiting lower toxicity. The PCL-PEOz drug-loaded targeted micelle can multi-target different subtypes of tumor cells and tumor-associated macrophages within the tumor. The synergistic effect after micelle administration achieves the goal of anticancer therapy combining targeted therapy and immunotherapy.

Specific embodiments of the present disclosure have been described above. It should be understood that the present disclosure is not limited to the particular embodiments described above, and that various variations or modifications may be made by a person skilled in the art within the scope of the claims without affecting the substantive content of the present disclosure.

What is claimed is:

1. A targeted drug-loaded micelle loaded with paclitaxel, comprising a non-targeted drug-loaded micelle loaded with paclitaxel and a first targeting group linked to a first micelle carrier, wherein
the non-targeted drug-loaded micelle has a first shell-core structure, and the first micelle carrier is prepared from poly(ε-caprolactone)-poly(2-ethyl-2-oxazoline) (PCL-PEOz), wherein a hydrophobic block of PCL and paclitaxel jointly form a core of the first shell-core structure, and a hydrophilic block of PEOz forms a shell of the first shell-core structure; and the first targeting group is linked via an amide bond formed between —NH₂ on a surface of the first targeting group and —COOH at a terminus of the PEOz on a surface of the shell.

2. The targeted drug-loaded micelle loaded with paclitaxel of claim 1, wherein in the non-targeted drug-loaded micelle loaded with paclitaxel, the paclitaxel is at a drug loading capacity of 2% to 10%.

3. The targeted drug-loaded micelle loaded with paclitaxel of claim 1, wherein the first targeting group is selected from the group consisting of a targeting antibody and a targeting peptide; and the targeting antibody comprises a full-length monoclonal antibody or a fragment of an antibody having targeting functionality.

4. The targeted drug-loaded micelle loaded with paclitaxel of claim 1, wherein the first targeting group comprises cetuximab; and a molar ratio of —COOH at the terminus of the PEOz to —NH₂ on the surface of the first targeting group is in a range of 0.1-10:1.

5. The targeted drug-loaded micelle loaded with paclitaxel of claim 4, further comprising hyaluronic acid; and a molar ratio of —NH₂ on the surface of the first targeting group to —COOH on a surface of hyaluronic acid is in a range of 1:1-20.

6. A method for preparing the targeted drug-loaded micelle loaded with paclitaxel of claim 1, comprising the following steps:
A1. mixing PCL-PEOz-COOH for preparing the first micelle carrier and paclitaxel to obtain a mixture, co-dissolving the mixture in an organic solvent to obtain a mixed material, adding water to the mixed material to form a mixed solution of the organic solvent and the water, and subjecting the mixed solution to ultrasonic treatment to form a homogeneous emulsion;
A2. removing the organic solvent from the homogeneous emulsion by rotary evaporation under reduced pressure or rotary evaporation under vacuum, and removing an unencapsulated drug to obtain the non-targeted drug-loaded micelle; and
A3. blending the non-targeted drug-loaded micelle with the first targeting group, adding a catalyst selected from the group consisting of 4-(4,6-dimethoxytriazin-2-yl)-4-methylmorpholine hydrochloride and a mixture of 1,3-dicyclohexylcarbodiimide and N-hydroxysuccinimide, and performing a chemical reaction to obtain the targeted drug-loaded micelle loaded with paclitaxel.

7. A combination preparation of paclitaxel and trabectedin, wherein the paclitaxel is in a form of the targeted drug-loaded micelle loaded with paclitaxel of claim 1.

8. The combination preparation of claim 7, wherein in the combination preparation, the trabectedin is in a form of a targeted drug-loaded micelle loaded with trabectedin.

9. The combination preparation of claim 8, wherein the targeted drug-loaded micelle loaded with trabectedin comprises a non-targeted drug-loaded micelle loaded with trabectedin and a second targeting group linked to a second micelle carrier;
the second micelle carrier is prepared from a material selected from the group consisting of poly(ε-caprolactone)-polyethylene glycol (PCL-PEG), poly(ε-caprolactone)-poly(2-ethyl-2-oxazoline) (PCL-PEOz), poly(lactic-co-glycolic acid)-polyethylene glycol (PLGA-PEG), and poly(lactic-co-glycolic acid)-poly(2-ethyl-2-oxazoline) (PLGA-PEOz);
the non-targeted drug-loaded micelle loaded with trabectedin has a second shell-core structure, wherein a hydrophobic block PCL or PLGA and trabectedin jointly form a core of the second shell-core structure, and a hydrophilic block PEG or PEOz forms a shell of the second shell-core structure; and
the second targeting group is linked via an amide bond formed between —NH₂ on a surface of the second targeting group and —COOH at a terminus of the PEG or the PEOz on a surface of the shell.

10. The combination preparation of claim 9, wherein in the non-targeted drug-loaded micelle loaded with trabectedin, the trabectedin is at a drug loading capacity of 2% to 10%; the second targeting group is a peptide of CSPGAK (Cysteine-Serine-Proline-Glycine-Alanine-Lysine) that specifically targets tumor-associated macrophages; and a molar ratio of —COOH at the terminus of the PEG or the PEOz to —NH₂ on the surface of the second targeting group is in a range of 0.1-10:1.

11. The method of claim 6, wherein in the non-targeted drug-loaded micelle loaded with paclitaxel, the paclitaxel is at a drug loading capacity of 2% to 10%.

12. The method of claim 6, wherein the first targeting group is selected from the group consisting of a targeting antibody and a targeting peptide; and the targeting antibody comprises a full-length monoclonal antibody or a fragment of an antibody having targeting functionality.

13. The method of claim 6, wherein the first targeting group comprises cetuximab; and a molar ratio of —COOH at the terminus of the PEOz to —NH₂ on the surface of the first targeting group is in a range of 0.1-10:1.

14. The method of claim 13, wherein the targeted drug-loaded micelle loaded with paclitaxel further comprises hyaluronic acid; and a molar ratio of —NH₂ on the surface of the first targeting group to —COOH on a surface of hyaluronic acid is in a range of 1:1-20.
